SEQUENCE LISTING (7 sequences)

SEQ ID NO: 1 - DNA, 20 bp, synthetic construct
  source 1..20: mol_type = other DNA; note = R1
cctcgatcccagatcggaga

SEQ ID NO: 2 - DNA, 20 bp, synthetic construct
  source 1..20: mol_type = other DNA; note = R2
ccgatctgtgatcgaggaga

SEQ ID NO: 3 - DNA, 10 bp, synthetic construct
  source 1..10: mol_type = other DNA
cacagatcgg

SEQ ID NO: 4 - DNA, 10 bp, synthetic construct
  source 1..10: mol_type = other DNA
gggatcgagg

SEQ ID NO: 5 - DNA, 10 bp, synthetic construct
  source 1..10: mol_type = other DNA
agatcggaag

SEQ ID NO: 6 - DNA, 17 bp, synthetic construct
  source 1..17: mol_type = other DNA
cctcgatcccagatcgg

SEQ ID NO: 7 - DNA, 17 bp, synthetic construct
  source 1..17: mol_type = other DNA
ccgatctgtgatcgagg

(19) United States
(12) Patent Application Publication
(10) Pub. No.: US 2025/0329418 A1
(43) Pub. Date: Oct. 23, 2025

(54) ADAPTER TRIMMING AND DETERMINATION IN NEXT GENERATION SEQUENCING DATA ANALYSIS

(71) Applicant: Element Biosciences, Inc., San Diego, CA (US)

(72) Inventor: Andrew ALTOMARE, Broomfield, CO (US)

(21) Appl. No.: 18/972,548

(22) Filed: Dec. 6, 2024

Related U.S. Application Data
(63) Continuation of application No. PCT/US2023/068053, filed on Jun. 7, 2023.
(60) Provisional application No. 63/350,290, filed on Jun. 8, 2022.

(51) Int. Cl. G16B 30/10; G16B 40/10

(52) U.S. Cl. CPC G16B 30/10; G16B 40/10

(57) ABSTRACT

Provided herein are system, apparatus, method, and/or computer program product embodiments, and/or combinations and sub-combinations thereof which enables adapter trimming and/or adapter determination during sequencing data analysis. Based on a plurality of match scores, one or more sequence alignments are selected. Each of the plurality of match scores may be based on a first number of matched bases and a second number of total bases. First and second consensus positions are generated from the one or more sequencing alignments. A trimming position is determined based on the first and second consensus positions and a first and second consensus match score.

### CROSS REFERENCE TO RELATED APPLICATIONS

This application is a continuation of PCT/US2023/068053 filed Jun. 7, 2023, which claims benefit to U.S. Provisional Patent Application No. 63/350,290, filed Jun. 8, 2022, which are hereby incorporated by reference in their entireties.

### TECHNICAL FIELD

This disclosure relates to adapter trimming in DNA sequencing reads, and particularly to adapter trimming/determination in pair-end DNA sequencing reads.

### BACKGROUND

In next-generation sequencing (NGS) or NGS-like applications such as sequencing by synthesis, sequencing by binding, or sequencing by avidity, trimming of adapter (or equivalently, adapters, primers, or linkers) from read data is a preprocessing step during sequencing data analysis. An adapter is a short, chemically synthesized, single-stranded or double-stranded oligonucleotide that is added to one or both ends of a sequencing read. The adapter can serve various functions including identifying the end(s) of the sequencing read and tethering the DNA fragment to a flow cell. Untrimmed adapters in the read data can look like errors to downstream data analysis. It is unknown in advance whether a read has sequenced into the adapter or not, and if so, how many bases of the adapter are included in the read. There is a need for adapter trimming methods that can accurately and efficiently determine a trimming position so that all the bases from the adapter(s) are trimmed without accidentally trimming any bases from the actual sequencing data.

### BRIEF SUMMARY

Provided herein are system, apparatus, method, and/or computer program product embodiments, and/or combinations and sub-combinations thereof which enables adapter trimming and/or adapter determination during sequencing data analysis. The sequencing reads can come from different sequencing technologies.

As a particular application of such, provided herein are embodiments of methods, systems, and media for adapter trimming and/or determination from sequencing reads, so that the sequencing results can be accurately and reliably generated.

Other embodiments of these aspects include corresponding computer systems, apparatus, and computer program products recorded on computer storage device(s), which, alone or in combination, configured to perform the actions or operations of the methods. For a computer system configured or to be configured to perform operations or actions, the computer system has installed on it software, firmware, hardware, or their combinations that in operation cause the computer system to perform the operations or actions. For a computer program product configured or to be configured to perform the operations or actions, the computer program product includes instructions that, when executed, by a hardware processor, cause the hardware processor to perform the operations or actions.

Further embodiments, features, and advantages of the present disclosure, as well as the structure and operation of the various embodiments of the present disclosure, are described in detail below with reference to the accompanying drawings.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the art(s) to make and use the embodiments.

FIG. 1 illustrates a block diagram of a system for DNA sequencing and generating sequencing reads, adapter trimming, and adapter determination, according to some embodiments.

FIG. 2 shows a schematic diagram of two example sequencing reads with adapters.

FIGS. 3A-3B show tables of sensitivity, specificity, phi coefficient for an embodiment of the adapter trimming method disclosed herein in comparison with other existing adapter trimming methods.

FIG. 4 illustrates a block diagram of a computer system for performing adapter trimming and adapter determination, according to some embodiments.

FIG. 5A illustrates a flow chart of a method for performing adapter trimming, according to some embodiments.

FIG. 5B illustrates a flow chart of a method for performing adapter determination, according to some embodiments.

FIGS. 6A-6E show comparison of adapter trimming results using methods disclosed herein and existing methods, according to some embodiments.

FIG. 7 is a schematic showing an example linear single stranded library molecule.

FIG. 8 is a schematic showing an example linear single stranded library molecule.

FIG. 9 is a schematic of various example configurations of multivalent molecules.

FIG. 10 is a schematic of an example multivalent molecule comprising a generic core attached to a plurality of nucleotide-arms.

FIG. 11 is a schematic of an example multivalent molecule comprising a dendrimer core attached to a plurality of nucleotide-arms.

FIG. 12 shows a schematic of an example multivalent molecule comprising a core attached to a plurality of nucleotide-arms, where the nucleotide arms comprise biotin, spacer, linker, and a nucleotide unit.

FIG. 13 is a schematic of an example nucleotide-arm comprising a core attachment moiety, spacer, linker, and nucleotide unit.

FIG. 14 shows the chemical structure of an example spacer (top), and the chemical structures of various example linkers, including an 11-atom Linker, 16-atom Linker, 23-atom Linker, and an N3 Linker (bottom).

FIG. 15 shows the chemical structures of various example linkers, including Linkers 1-9.

FIG. 16 shows the chemical structures of various example linkers joined/attached to nucleotide units.

FIG. 17 shows the chemical structures of various example linkers joined/attached to nucleotide units.

FIG. 18 shows the chemical structures of various example linkers joined/attached to nucleotide units.

FIG. 19 shows the chemical structures of various example linkers joined/attached to nucleotide units.

FIG. 20 shows the chemical structure of an example biotinylated nucleotide-arm. In this example, the nucleotide unit is connected to the linker via a propargyl amine attachment at the 5 position of a pyrimidine base or the 7 position of a purine base.

FIG. 21 provides a schematic illustration of one embodiment of the low binding solid supports of the present disclosure.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, method, and/or computer program product embodiments, and/or combinations and sub-combinations thereof which enables adapter trimming and/or adapter determination of sequencing reads for generating accurate sequencing results. The techniques disclosed herein can be used on sequencing reads obtained from various imaging and/or sequencing techniques. The techniques can be used on sequencing reads obtained from various sequencing samples including two dimensional (2D) and/or three-dimensional (3D) samples. Techniques disclosed herein are useful for excluding adapter(s) in sequencing read results in NGS, and NGS sequencing reads will be used as the primary example herein for describing the application of these techniques. However, such adapter trimming technologies may also be useful in other applications.

In DNA sequencing, identifying the centers of clusters or polonies (which are often formed on beads) is sometimes referred to as primary analysis. Primary analysis can include base calling in which bases in a sequencing read are identified to form an orderly sequence of different bases, such as adenine (A), cytosine (C), guanine (G), and thymine (T). Subsequent to primary analysis and more specifically, to base calling, embodiments of the techniques disclosed herein can be used for adapter trimming of sequencing reads. A variety of algorithms exist for adapter trimming. These existing algorithms suffer from various shortcomings. For example, existing algorithms are not adapted to working directly with sequencing reads output from sequencers, and can take a long time, on a scale of at least a couple hours, to trim the adapters. As another example, adapter trimming using an alignment accuracy as a threshold may not provide reliable results because a 0.8 accuracy threshold may be satisfied by 4 matches out 5 bases, but not 32 matches out of 64 bases. However, the likelihood of randomly matching 32 of 64 bases is lower than 4 of 5 bases. Additionally, when sequencing data includes indels, existing methods rely on indel processing that is complex and additional trimming operations which exert extra computational costs and time consumption to trim an adapter when an indel is present.

Embodiments of the technologies disclosed herein advantageously utilize random matching and probability distribution like binomial distribution to replace the accuracy threshold in existing methods, and determine the likelihood that the matched bases do not occur by random chance. Embodiments of the techniques disclosed herein advantageously work on sequencing reads directly in binary format after they are outputted from sequencers, so that it eliminates the need to process the output from sequencers to other formats and saves computational time in doing binary arithmetic in adapter trimming operations. Embodiments of the adapter trimming techniques disclosed herein utilize 2, 3, or 4 alignments obtained from the forward and reverse reads in paired-end sequencing and eliminate the need for additional indel processing since the indel handling is intrinsic to the trimming methods, such that an indel in the insert or the adapter only affects some but not all the alignments being used, and the trimming position can still be accurately identified. That way, the technologies allow improved accuracy over existing methods with a significant reduction in computational complexity and computational time, e.g., reduction from a couple of hours to less than 10 minutes or even a couple of seconds.

Embodiments of the techniques disclosed herein also advantageously determine the adapter sequences based on the determination of possible adapter positions and similarity of candidate adapter sequences. The determination of adapter sequences using embodiments of the methods disclosed herein may be used to facilitate sequencing applications using different sequencing kits or chemistry that relies on different adapters. The determination of adapter sequences using embodiments of the methods disclosed herein may be utilized for checking adapter sequences (e.g., manually entered in sequencing parameters) for accuracy and reliability before any subsequent analysis occurs. Embodiments of the adapter determination methods disclosed herein may also advantageously facilitate accurate adapter trimming thus improving adapter-induced error(s) in secondary analysis.

### Sequencing Systems

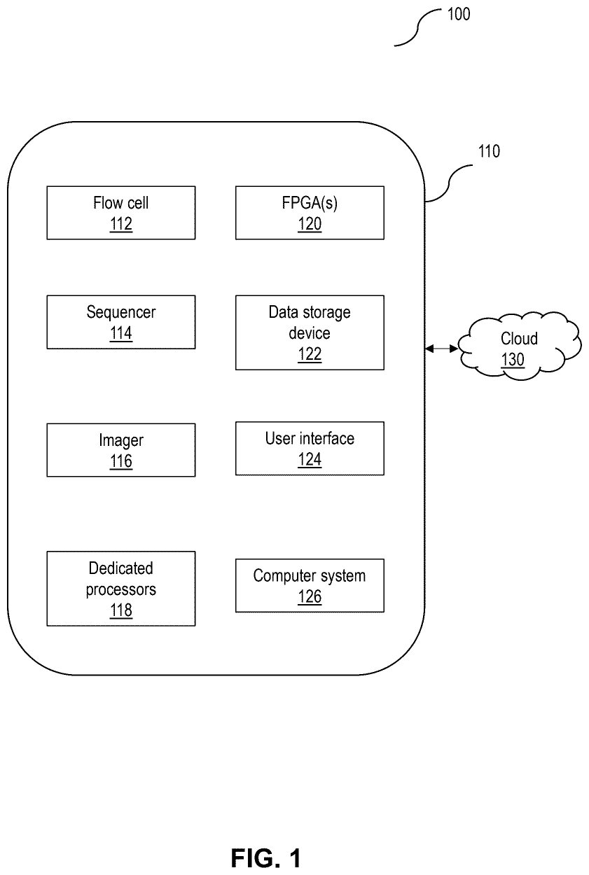

FIG. 1 illustrates a block diagram of a computer-implemented system 100 for generating sequencing reads, performing adapter trimming and/or adapter determination, according to one or more embodiments disclosed herein. The system 100 has a sequencing system 110 that includes a flow cell 112, a sequencer 114, an imager 116, data storage 122, and user interface 124. The sequencing system 110 may be connected to a cloud 130. The sequencing system 110 may include one or more of dedicated processors 118, Field-Programmable Gate Array(s) (FPGAs) 120, and a computer system 126.

In some embodiments, the flow cell 112 is configured to capture DNA fragments and form DNA sequences for base-calling on the flow cell. The flow cell 112 can include the support as disclosed herein. The support can be a solid support. The support can include a surface coating thereon as disclosed herein. The surface coating can be a polymer coating as disclosed herein.

A flow cell 112 can include multiple tiles or imaging areas thereon, and each tile may be separated into a grid of subtiles. Each subtile can include a plurality of clusters or polonies thereon. As a nonlimiting example, a flow cell can have 424 tiles, and each tile can be divided into a 6×9 grid, therefore 54 subtiles. The flow cell image as disclosed herein can be an image including signals of a plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during an imaging or sequencing cycle using the imager 116. In some embodiments, each tile may include millions of polonies or clusters. As a nonlimiting example, a tile can include about 1000 to 10 millions of clusters or polonies. Each polony can be a collection of many copies of DNA fragments. In some embodiments, each tile or subtile may include millions of polonies or clusters. As a nonlimiting example, a tile may include 1,000 to 10 million of clusters or polonies. Each polony may be a collection of many copies of DNA fragments. In some embodiments, a flow cell image may be an image that includes all the tiles and approximately all signals thereon. The flow cell image may be acquired from a channel during an imaging or sequencing cycle using the imager 116.

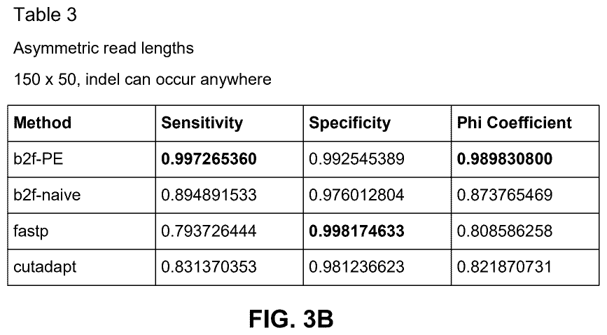

In cases where in situ samples, e.g., cells or tissues are immobilized on the support or flow cell, the flow cell images may be at multiple z levels which are orthogonal to the image plane of the flow cell images. In particular, for three dimensional (3D) samples, e.g., cells, tissues, or other in situ samples, the flow cell images can include multiple z-levels in order to cover the whole sample(s) in 3D. The z axis can extend from the objective lens of the optical system disclosed herein to the support, e.g., flow cell. The axial axis can be orthogonal to the image plane of the flow cell images. Each z level of flow cell images may be separated from the adjacent z level(s) for a predetermined distance, for example, for about 0.1 μm to about 15 ums. Each z level of flow cell images may be separated from the adjacent level(s) for 1 μm to 10 ums. At each z-level, a flow cell image can be acquired from one or more sequencing cycles and/or one or more channels. Each flow cell image may include in its field of view at least part of one or more tiles or subtiles of the flow cell. FIG. 3 shows a portion of a flow cell 112 with multiple tiles 290. The image plane is defined by the x and y axis. And the z axis is orthogonal to the x-y plane. Although the flow cell images, samples, and the axial axis are described in a Cartesian coordinate system as shown in FIG. 3, any other coordinate systems can be used to define spatial locations and relationships herein. Other coordinate systems can include but are not limited to the polar coordinate system, cylindrical, or spherical coordinate systems.

The sequencer 114 may be configured to flow a nucleotide mixture onto the flow cell 112, cleave blockers from the nucleotides in between flowing steps, and perform other steps for the formation of the DNA sequences on the flow cell 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some embodiments, the sequencer 114 and the flow cell 112 may be configured to perform various sequencing methods disclosed herein, for example, sequencing-by-avidite.

For example, each nucleotide base may be assigned a color. Different types of nucleotides can have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

The imager 116 may be configured to capture images of the flow cell 112 after each flowing step. In an embodiment, the imager 116 is a camera configured to capture digital images, such as an active pixel sensor (CMOS) or a charge coupled device (CCD) camera. The camera may be configured to capture images at the wavelengths of the fluorescent elements bound to the nucleotides. The images can be called flow cell images.

In some embodiments, the imager 116 can include one or more optical systems disclosed herein. The optical system(s) can be configured to capture optical signals from the flow cell and generate corresponding digital images thereof. The digital images can then be used for base calling.

In an embodiment, the images of the flow cell may be captured in groups, where each image in the group is taken at a wavelength or in a spectrum that matches or includes only one of the fluorescent elements. In another embodiment, the images may be captured as single images that captures all of the wavelengths of the fluorescent elements.

The resolution of the imager 116 controls the level of detail in the flow cell images, including pixel size. In existing systems, this resolution is very important, as it controls the accuracy with which a spot-finding algorithm identifies the polony centers. In some embodiments, the image resolution of flow cell images disclosed herein can be about 10 nanometers (nms) to a couple of hundreds of nms or greater. One way to increase the accuracy of spot finding is to improve the resolution of the imager 116 (e.g., by incorporating a higher resolution camera), or improve the processing performed on images taken by imager 116. Detecting polony centers in pixels other than those detected by a spot-finding algorithm can be performed. These methods can allow for improved accuracy in detection of polony centers without increasing the resolution of the imager 116. The resolution of the imager may even be less than existing systems with comparable performance, which may reduce the cost of the sequencing system 110. In some aspects, the resolution of the imager may be the same as existing systems but achieve superior performance as compared to those existing systems due to the image processing.

The image quality of the flow cell images controls the base calling quality. One way to increase the accuracy of base calling is to improve the imager 116, or improve the processing performed on images taken by imager 116 to result in a better image quality.

After base calling is performed, with the option of certain processing on base calling results, sequencing reads can be outputted from the system to the cloud 130 or to a computer system 400. The sequencing read(s) herein can be a forward read (R1), a reverse read (R2), or both. The sequencing reads herein can be any orderly sequence of bases of A, T,C, and G.

In some embodiments, the sequencing reads can be directly communicated to the computer system 126 for adapter trimming.

These adapter trimming methods can be advantageously performed in parallel in the computer system 400, without interference with or delay of existing sequencing workflow of the system 100. The results of adapter trimming can be made available for generating sequencing results for users. Some or all of the operations disclosed herein can be advantageously performed by the FPGA(s) and data can be communicated between the CPU(s) and FPGA(s) to reduce the total operational time from methods operating without the FPGA(s). Furthermore, instead of handling the alignment in standard format like A, T, C, G, the methods disclosed herein advantageously work on sequencing data in binary format, with each base represented by a couple of binary bits, to significantly speed up the adapter trimming process, so that the trimming can be completed on a range from a couple of seconds to a couple of minutes depending on the size of the data.

The operations or actions disclosed herein may be performed by the dedicated processors 118, the FPGA(s) 120, the computing system 126, or a combination thereof. One or more operations or actions in methods 500, 600 disclosed herein may be performed by the dedicated processors 118, the FPGA(s) 120, the computing system 126, or a combination thereof. In some embodiments, which operations or actions are to be performed by performed by the dedicated processors 118, the FPGA(s) 120, the computing system 126, or their combinations can be determined based on one or more of: a computation time for the specific operation(s), the complexity of computation in the specific operation(s), the need for data transmission between the hardware devices, or their combinations.

The computing system 126 can include one or more general purpose computers that provide interfaces to run a variety of program in an operating system, such as Windows™ or Linux™. Such an operating system typically provides great flexibility to a user.

In some embodiments, the dedicated processors 118 may be configured to perform operations in the methods of adapter trimming. They may not be general-purpose processors, but instead custom processors with specific hardware or instructions for performing those steps. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the steps are performed and allow for real time processing.

In some embodiments, the FPGA(s) 120 may be configured to perform operations of the adapter trimming methods herein. An FPGA is programmed as hardware that will only perform a specific task. A special programming language may be used to transform software steps into hardware componentry. Once an FPGA is programmed, the hardware directly processes digital data that is provided to it without running software. The FPGA instead uses logic gates and registers to process the digital data. Because there is no overhead required for an operating system, an FPGA generally processes data faster than a general-purpose computer. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 120 may be configured to perform the steps in parallel. For example, a number of FPGA(s) 120 may be configured to perform a processing step for an image, a set of images, a subtile, or a select region in one or more images. Each FPGA(s) 120 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over conventional systems may need to store the data before it may be processed, which may require more memory or accessing a computer system located in the cloud 130.

In some embodiments, the data storage 122 is used to store information used in the adapter trimming methods. This information may include the sequencing reads and adapters themselves or information (e.g., pixel intensities, colors, etc.) that can be used during the adapter trimming operations. For example, probability look-up tables as disclosed herein can be save in the data storage 122. The DNA sequences determined after adapter trimming may be stored in the data storage 122. Compressed and/or uncompressed sequencing data may be stored in the data storage. The FASTQ file may also be stored in the data storage 122.

The user interface 124 may be used by a user to operate the sequencing system or access data stored in the data storage 122 or the computer system 126.

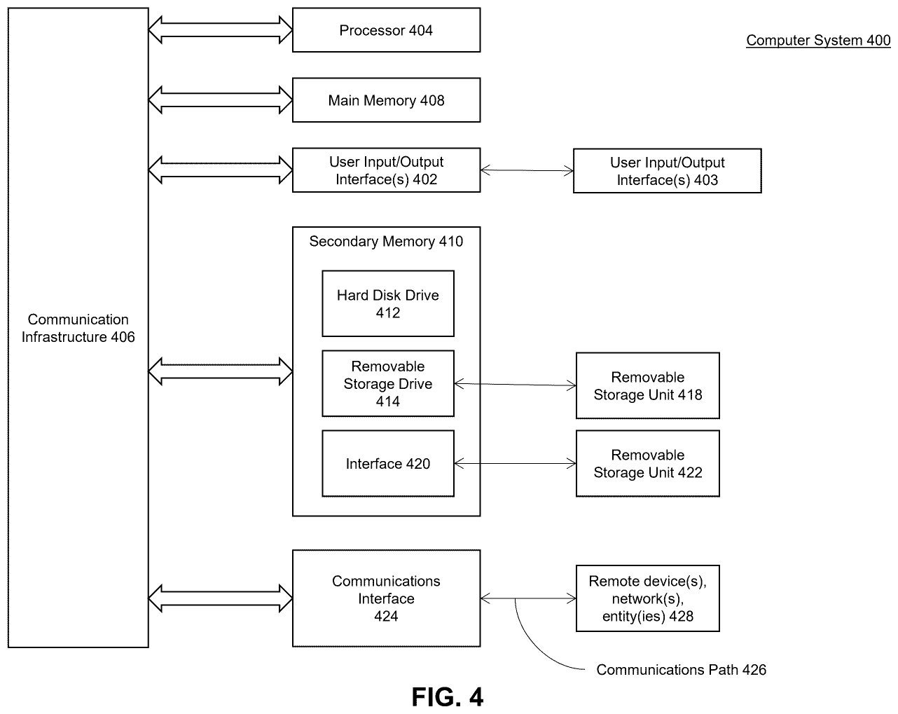

The computer system 126 may control the general operation of the sequencing system and may be coupled to the user interface 124. It may also perform steps in adapter trimming and its preceding operations, and/or subsequent operations, such as base calling, demultiplexing, etc. In some embodiments, the computer system 126 is a computer system 400, as described in more detail in FIG. 4. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish necessary processing described above. In some embodiments, when these elements are present together, the processing tasks are split between them. For example, the FPGA(s) 120 may be used to perform some portion or all of: the operations preceding to adapter trimming, adapter trimming, and the subsequent operations, while the computer system 126 may perform other processing functions for the sequencing system 110. Those skilled in the art will understand that various combinations of these elements will allow various system embodiments that balance efficiency and speed of processing with cost of processing elements.

The cloud 130 may be a network, remote storage, or some other remote computing system separate from the sequencing system 110. The connection to cloud 130 may allow access to data stored externally to the sequencing system 110 or allow for updating of software in the sequencing system 110.

### Adapter Trimming

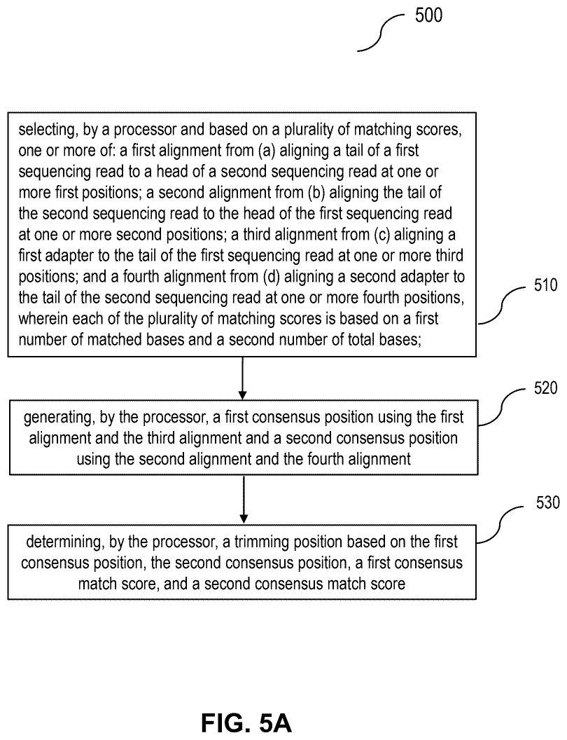

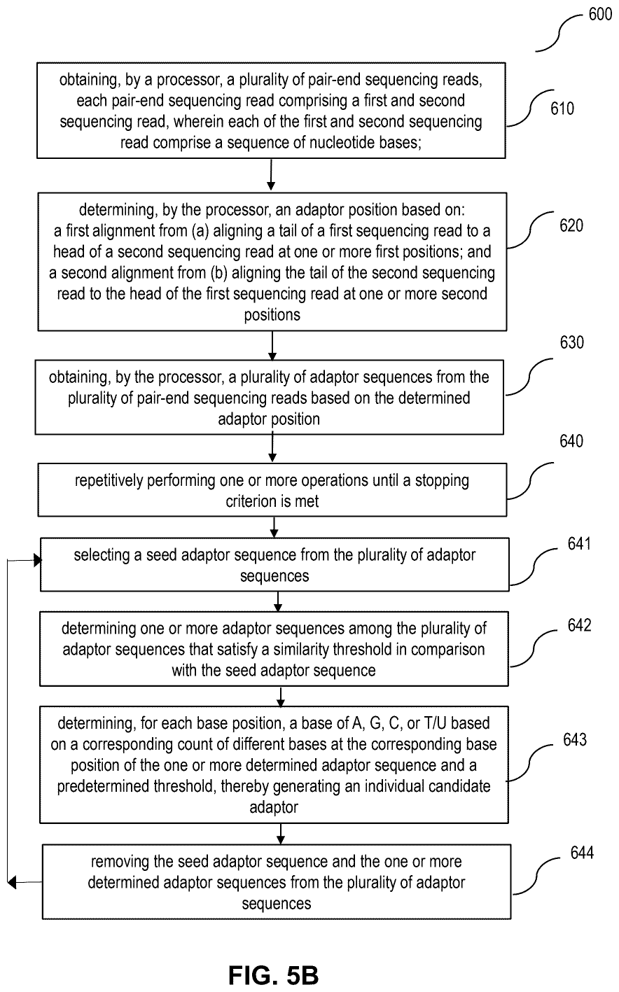

FIG. 5A shows a flow chart of an example embodiment of the computer-implemented method 500 for adapter trimming of sequencing reads. The method 500 can include some or all of the operations disclosed herein. The operations may be performed in but is not limited to the order that is described herein.

The method 500 can be performed by one or more processors disclosed herein. In some embodiments, the processor can include one or more of: a processing unit, an integrated circuit, or their combinations. For example, the processing unit can include a central processing unit (CPU) and/or a graphic processing unit (GPU). The integrated circuit can include a chip such as a field-programmable gate array (FPGA). In some embodiments, the processor can include the computing system 400.

In some embodiments, some or all operations in method 500 can be performed by the FPGA(s). In embodiments when some operations are performed by FPGA(s), the data after an operation performed by the FPGA(s) can be communicated by the FPGA(s) s to the CPU(s) so that CPU(s) can perform subsequent operation(s) in method 500 using such data. Similarly, data can also be communicated from the CPU(s) to the FPGA(s) for processing by the FPGA(s). In some embodiments, all the operations in method 500 can be performed by CPU(s). Alternatively, the operations performed by CPU(s) can be performed by other processors such as the dedicated processors, or GPU(s). In some embodiments, all the operations in method 500 can be performed by FPGA(s).

In some embodiments, the method 500 is configured to trim, clip, or otherwise remove adapters from sequencing reads. The sequencing reads can be determined by analysis of flow cell images generated by the system 100. In some embodiments, the method 500 is performed after primary analysis is performed and sequencing reads are generated from the system 100. In some embodiments, the method 500 is performed after demultiplexing. In some embodiments, the method 500 is performed before demultiplexing.

In some embodiments, the methods 500 is performed after cycle N has been completed, while sequencing, image acquisition of cycle N+1 is yet to be performed. In some embodiments, the methods 500 is performed after the entire sequence run is completed. In some embodiments, cycle N is the current cycle. N can be any non-zero integer. In some embodiments, cycle N is the cycle after the cycles corresponding to adapter(s). In some embodiments, cycle N can be determined based on the adapter lengths and/or sequence insert length. The sequence insert is the part that contains DNA fragment of interest from sequencing sample(s). In some embodiments, cycle N can be determined if insert lengths are within a range, e.g., 100 to 150. For example, N can be any integer from 30 to 300 or 20 to 400. In some embodiments, N is not the last cycle in the sequencing run. In some embodiments, N is in the first half or first one third of the total number of sequencing cycles in the sequencing run. In some embodiments, the method 500 is performed while the sequencing run is being performed. In some embodiments, the method 500 is performed and the result of adaptor trimming or adapter determination may be used to determine if the sequencing run in progress should be stopped or not if the adapter lengths and/or sequence insert length is pre-known before cycle N.

The flow cell images can be acquired using the optical system disclosed herein, from one of the 1, 2, 3, 4, or more channels of the imager 116. Each flow cell image can include one or more tiles (imaging areas), and each tile can be divided into multiple subtiles. Each subtile can include a plurality of polonies. Each subtile can include multiple regions with each region including a number of polonies. The flow cell image as disclosed herein can be an image that is acquired using a flow cell 112 as shown in FIG. 1.

The flow cell 112 may include sample(s) immobilized thereon. The sample(s) may include a plurality of nucleic acid template molecules. The sample(s) may include a 2D or a 3D volumetric sample. The nucleic acid template molecules may be distributed randomly or in various patterns on the flow cell 112. In some embodiments, the plurality of polonies or clusters herein may be extracted from specific regions of a tile, e.g., each subtile. With each subtile, the polonies may be extracted with a predetermined pattern or randomly.

In some embodiments, the polonies or clusters being sequenced in a flow cycle may have a certain nucleotide diversity. The nucleotide diversity of a population of nucleotide acid molecules, e.g., polonies or clusters, can refer to the relative proportion of nucleotides A, G, C, and T/U that are present in each flow cycle. An optimally high or balanced diversity data can generally have approximately equal proportions of all four nucleotides represented in each flow cycle of a sequencing run. A low or unbalanced diversity data can generally include a high proportion of certain nucleotides and low proportion of other nucleotides in some flow cycles of a sequencing run, e.g., less than 10% of the total number of all 4 nucleotides. As a result, images corresponding to the high portion of certain nucleotides can have more signal spots (polonies or clusters) than images corresponding to the low portion of certain nucleotides. As an example of low or unbalanced diversity data in a flow cycle, the bases A, T, C, G can be about 1%, about 2%, about 1%, and about 95%, respectively, of the total number of polonies, in a certain flow cycle. As another example of low or unbalanced diversity data, the bases A, T, C, G in polonies at multiple flow cycles can be about 2%, about 5%, about 10%, and about 83%, respectively. In embodiments where low or unbalanced diversity data is present in a particular cycle and is imaged for sequencing analysis, base calling may be prone to errors, and existing technologies for adapter trimming or determination may fail because errors in base calling may cause insertion, deletion of nucleotide bases in the sequence reads.

In addition to the base biases affecting diversity, plexity can also be a factor that affects image registration. In general, plexity can indicate source(s) of the sample. A uniplex sample may include DNA fragments or molecules from a same sample region in a genome or a same sample source. A multiplex sample may include DNA fragments or molecules from different sample sources, e.g., liver, kidney, heart, cancerous tissue, etc., or from one or more sample regions in the genome. When plexity is lower than a number, e.g., 8 or 16, the signal may be of low diversity. For example, in a 2-cycle sequence, all polonies are of AT or TG or GC or CA. Every base is 25% of the total number of bases in that cycle, but its plexity is less than 8, and the sequence is not all random. The method 500 may accurately trim and/determine the adapters even if the sequencing reads are generated from low diversity data.

In some embodiments, the method 500 can include an operation 510 of selecting one or more alignments from:
a first alignment from (a) aligning a tail of a first sequencing read 212 to a head of a second sequencing read 221 at one or more first positions;
a second alignment from (b) aligning the tail of the second sequencing read 222 to the head of the first sequencing read 211 at one or more second positions;
a third alignment from (c) aligning a first adapter 213 to the tail of the first sequencing read 212 at one or more third positions; and
a fourth alignment from (d) aligning a second adapter 223 to the tail of the second sequencing read 222 at one or more fourth positions.

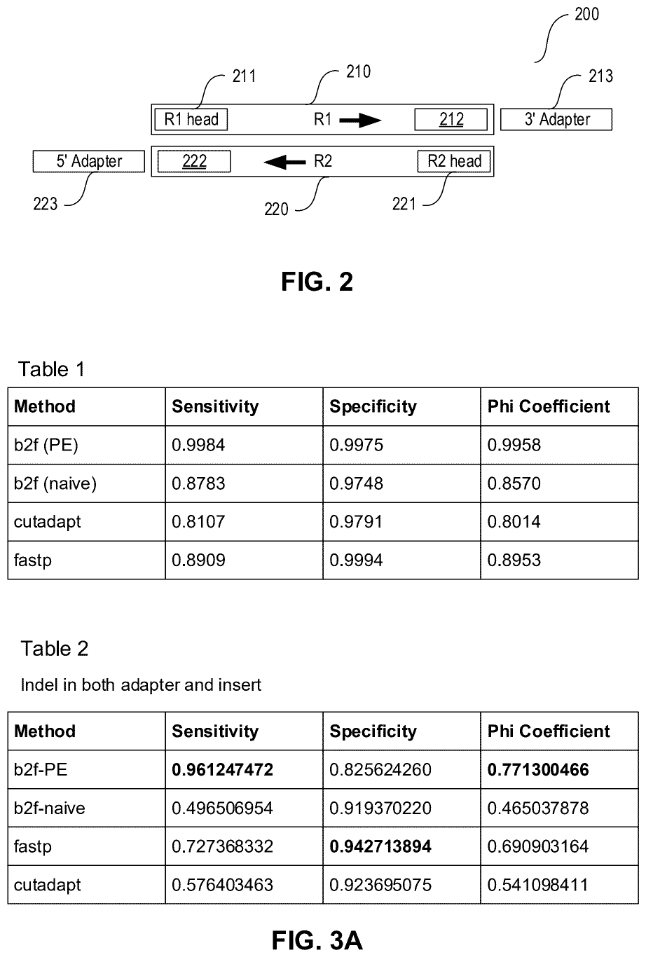

FIG. 2 shows a schematic diagram of an example sequencing read disclosed herein, according to some embodiments. The sequencing read 200 can be generated by the system 100 and communicated to a processor within the system 100. Alternatively, the sequencing read can be outputted by the system 100 to a cloud or a processor, e.g., computer system 400, external to the system 100.

In some embodiments, each of the first sequencing read 210, the second sequencing read 220, the head of the first sequencing read 211, the head of the second sequencing read 221, the tail of the first sequencing read 212, the tail of the second sequencing read 222, the first adapter 213, and the second adapter 223 comprises a sequence of nucleotide bases. The sequence of the nucleotide bases can be an orderly sequence and each base can be one of the four different bases, e.g., A, G, C, T/U. The sequence of bases can be of low diversity so that one or more of the bases only appear in less than 10% as disclosed herein.

The sequencing reads can be generated from a pair-end sequencing run, e.g., by primary analysis. The sequencing reads may have not been demultiplexed yet. In some embodiments, the sequencing reads may have been demultiplexed. Each run may include multiple number of paired sequencing reads 200. For example, a single run can include various number of sequencing reads, e.g., 100 to 10⁸ sequencing reads. The amount of sequencing data, e.g., 100,000 pair end sequencing reads with each read has a length in the range from about 30 bases to about 300 bases, and its processing, handling, and recording demands methods like those disclosed herein that are computationally efficient and time effective to facilitate sequencing analysis, e.g., secondary analysis. In some embodiments, some or all of the sequencing reads 200 in a sequence run can be processed using methods 500 here for adapter trimming.

The sequencing read 200 can include a forward read (R1) 210, and a reverse read (R2)220. Both the forward read 210 and the reverse read 220 can include an insert having a head portion 211, 221 and a tail portion 212, 222. The reads 210, 220 can have an adapter 213, 223 ligated or otherwise attached to the tail 212, 222. The insert is the sequence of bases of interest. In some embodiments, the reads 210, 220 can have an adapter ligated or otherwise attached to the head 212, 222 (not shown). In some embodiments, the reads 210, 220 can have an adapter ligated or otherwise attached to both the head 211, 221 and the tail 212, 222 (not shown). The first adapter 213 can be a 3′ adapter and the second adapter 223 can be a 5′ adapter.

In some embodiments, the insert in reads 210, 220 can have various numbers of A, G, C, T/U bases ranging from about 8 bases to about 500 bases. In some embodiments, the insert in reads 210, 220 can have any number of bases ranging from about 16 bases to about 500 bases. In some embodiments, the insert in reads 210, 220 can have any number of bases ranging from about 16 bases to about 500 bases. In some embodiments, the insert in reads 210, 220 can have any number of bases ranging from about 32 bases to about 400 bases. In some embodiments, the insert in reads 210, 220 can have any number of bases ranging from about 64 bases to about 250 bases. In some embodiments, the insert in the forward read 210 and the insert in the reverse read 220 can be of the same length. In some embodiments, the insert in the forward read 210 and the insert in the reverse read 220 can be of different length.

In some embodiments, the head 211, 221 and/or the tail 212, 222 can have a number of bases in the range of about 8 bases to about 200 bases. In some embodiments, the head 211, 221 and/or the tail 212, 222 can have a number of bases in the range of about 16 bases to about 150 bases. In some embodiments, the head 211, 221 and/or the tail 212, 222 can have a number of bases in the range of about 16 bases to about 80 bases. In some embodiments, the head 211, 221 and/or the tail 212, 222 can have a number of bases in the range of about 16 bases to about 64 bases. In some embodiments, the head 211, 221 and/or the tail 212, 222 have a number of bases in the range of about 16 bases to about 32 bases. In some embodiments, the head 211, 221 and/or the tail 212, 222 have at least 16 bases. In some embodiments, each of the head 211, 221 and the tail 212, 222 can be of a different number of bases. In some embodiments, two or more of the head 211, 221 and the tail 212, 222 can have a same number of bases.

In some embodiments, the first adapter 213 and/or the second adapter 223 has about 4 bases to about 100 bases. In some embodiments, the first adapter 213 and/or the second adapter 223 has about 4 bases to about 64 bases. The first adapter or the second adapter can have about 16 bases to about 64 bases. The first adapter or the second adapter can have about 16 bases to about 40 bases. In some embodiments, the first adapter 213 and/or the second adapter 223 has about 30, about 31, about 32, about 33, or about 34 bases. In some embodiments, the first adapter 213 and/or the second adapter 223 has 30, 31, 32, 33, or 34 bases. The first adapter 213 and/or the second adapter 223 can have at least 16 bases. The first adapter 213 and the second adapter 223 can have a same number of bases. In some embodiments, the first adapter 213 and the second adapter 223 can have different numbers of bases. For example, the first adapter can have more bases than the second adapter 223.

In some embodiments, the operation 510 can include selecting all four alignments, i.e., the first alignment from (a), the second alignment from (b), the third alignment from (c), and the fourth alignment from (d) as disclosed herein.

In some embodiments, selecting a first alignment from (a) aligning a tail of a first sequencing read 212 to a head of a second sequencing read 221 at one or more first positions comprises an operation of obtaining the reverse complement bases of the head of the second sequencing read. Subsequent to that, the method 500 can include an operation of aligning the reverse complement bases of the head of the second sequencing read to the tail of the first sequencing read at a position, and calculating a match score at the position, and then move the reverse complement bases of the head of the second sequencing read relative to the tail of the first sequencing read to a next position, by a base, and repeat the calculation of the match score at the next position. Such moving relative to each other and calculation of match scores can be repeated to cover all positions from aligning 1 base to aligning all possible bases of the tail of the first sequencing read to the head of the second sequencing read. Subsequently, the first alignment can be selected based on the match scores at the one or more first alignment positions.

For example, in a sequencing read with 2×20 bases, R1 is CCTCGATCCCAGATCGGAGA, and R2 is CCGATCTGTGATCGAGGAGA, which is the reverse compliment of R1 with 1 sequencing error. The last 3 bases are bases from the adapter(s). In this embodiment, the head of R2 read is the reverse complement of the first 10 bases of R2. So, R2 head is CACAGATCGG. The R2 head is aligned with the tail of R1, and moves base by base toward the head of R1 (moves from right to left) at different alignment positions with 1 base aligned, 2 bases aligned, etc. When there are 10 bases aligned, there are multiple alignment positions as the R2 head can keep moving left so that it aligns with all different options of 10 bases in R1. A match score is calculated at each different alignment position as R2 head keeps moving base by base. A total number of 20 or more match scores can be calculated as R2 heads moves base by base toward the head of R1. The first alignment is determined when R2 head aligns with the 8th base to the 17th base in R1 as:

R1 read:
CCTCGATCCCAGATCGGAGA
R2 head:
CACAGATCGG

The first alignment is determined based on the match scores at all the possible alignment positions. With the first alignment, there are a total number 10 aligned bases, 9 out of 10 are matched bases, so that n=10, M=9.

The match score is based on a first number of matched bases at the first alignment and a second number of total aligned bases. In this example, there are 9 matched bases out of a total number of 10 aligned bases. In some embodiments, the match score is based on a probability that the first number of matched bases does not occur randomly. In some embodiments, the match score is based on a probability that the first number of matched bases over a total number of aligned bases does not occur randomly.

In some embodiments, the match score comprises a probability as a constant number subtracting a cumulative distribution function (CDF) of a variable, m. The variable, m, can have different distributions such as a binomial distribution. The binomial distribution can be determined by a total number of aligned bases, n, and a probability of randomly matching a base in an alignment, p. In this particular example, p is randomly matching a base to 4 different bases, so, p is 0.25, and the distribution is binomial (n,0.25). The CDF of variable m is P (m>=M), wherein M is the number of matched bases. In some embodiments, the match score is C—P (m>=M), wherein C is a constant, M is a first number of matched bases, m is a variable with a binomial distribution, binomial (n,p), n is a number of total aligned bases, and p is a probability of randomly matching a base to a number of different bases. When the constant number is set as 1 and the match score is greater than about 0.99000, about 0.99900, about 0.99990, or about 0.99999. In some embodiments, the match score is greater than 0.99000, 0.99900, 0.99990, or 0.99999.

In some embodiments, the variable, m, can have a binomial distribution. In some embodiments, the base composition can be estimated, and a multinomial distribution can be used. In some embodiments, a different binomial can be used in each cycle so that the binomial distribution is scaled by an expected or learned per-cycle error rate. When m has a distribution that is different from the binomial distribution as disclosed herein, the match score and consensus match score disclosed herein can be similarly determined by replacing the binomial distribution with other distributions.

In some embodiments, the match score can be selected as the highest among all match scores at possible alignment positions. In some embodiments, the match score can be selected as a score that is higher than a predetermined threshold. In some embodiments, the match score can be the highest among all match scores and also satisfying a predetermined threshold. In this particular example, the match score of the first alignment is the highest among all match scores at possible alignment positions of the tail of R1 read and the head of R2 read. The match score is calculated as 1-P (m>=9), where m has a distribution of Binom (10, 0.25), and the score has a value of 0.999970436.

Similarly, as the first alignment, the second alignment can be determined. R1 head can be similarly defined as the reverse complement of the first 10 bases of R1. In this particular example, R1 head is GGGATCGAGG. The second alignment with highest match score is aligning R1 head from 8th to 17th bases of R2 read, as:

R2 read:
CCGATCTGTGATCGAGGAGA
R1 head:
GGGATCGAGG

The match score is calculated as 1-P (m>=9), where m˜Binom (10, 0.25), and the score has a value of 0.999970436.

Similarly, the third alignment can be selected from (c) aligning the first adapter 213 to the tail of the first sequencing read 212 at one or more third positions. In this particular example, the third alignment with highest match score is aligning the first adapter from the 10th base to the 20^{th} base of R1 read as:

R1 read:
CCTCGATCCCAGATCGGAGA
1st adapter:
AGATCGGAAG

The match score is calculated as 1-P (m>=8), where m˜Binom (10, 0.25), and the score has a value of 0.999584197. The third alignment is spurious and does not indicate the trimming position.

The fourth alignment from (d) aligning the second adapter 223 to the tail of the second sequencing read 222. In this particular example, the fourth alignment with highest match score is aligning the second adapter from the 17^{th} base to the 20^{th} base of R2 read as:

R2 read:
CCGATCTGTGATCGAGGAGA
2nd adapter:
AGATCGGAAG

The match score is calculated as 1-P (m>=3), where m˜Binom (3, 0.25), and the score has a value of 0.984375. The fourth alignment indicates the correct trimming position.

In some embodiments, the method 500 can include an operation of obtaining the first sequencing read with a first adapter and a second sequencing read with a second adapter. The reads can be obtained from the sequencing system 110. More specifically, it may be directly from the sequencer 114, the data storage 122, or any processors such as 120, 118, and/or 126. Alternatively, the reads can be obtained from a cloud 130 that is external to the sequencing system 110.

In some embodiments, the sequencing reads are obtained directly so that the format of the bases in the reads does not require additional formatting or preprocessing. For example, the reads can be directly obtained in binary format. Each base in one or more of: the head of the first sequencing read; the head of the second sequencing read; the tail of the first sequencing read; the tail of the second sequencing read; the first adapter; and the second adapter can be expressed as an integer. Such integer can be a bitwise integer with a number of binary bits. For example, each base can be expressed as a binary number with 4 bits.

In some embodiments, with the bases as binary number with multiple bits, the reverse compliment, alignment of bases, and determination of matched bases in the operations disclosed herein can be advantageously performed using bitwise arithmetic. Bitwise arithmetic can significantly speed up the operations disclosed herein comparing with same operations but using different format of bases. Bitwise arithmetic can also significantly reduce computational time in comparison to existing methods of adapter trimming. For example, the operations here take a couple of seconds to a couple of minutes to do adapter trimming while existing methods may take a couple hours to trim the adapters with reduced accuracy than the methods disclosed herein. In some embodiments, the method 500 can include an operation 520 of generating a first consensus position using the first alignment and the third alignment and a second consensus position using the second alignment and the fourth alignment.

In some embodiments, the operation 520 comprises determining a first adapter position from the first alignment, and a second adapter position from the third alignment. The first or second adapter position can indicate at what position the insert of the reads end and the adapter bases starts. For example, the first adapter position can be that the adapter starts at the 17^{th} base of the R1 read.

In response to determining that the first adapter position agrees with the second adapter position, the operation 520 includes determining the first consensus position as the first adapter position.

In response to determining that the first adapter position disagrees with the second adapter position, either the first adapter position or the second adapter position can be the first consensus position. In this particular example, the first adapter position indicates that last 3 bases of R1 read is from the adapter. The second adapter position indicates that 10 bases at the end of R1 read is from the adapter. The two adapter positions from the first alignment and the third alignment disagree with each other. The first consensus position can be either the first adapter position or the second adapter position. Further steps as described herein can be taken to determine which one of the first or second adapter position can be the first consensus position.

The method 500 can include an operation of determining a first consensus match score based on the first alignment and the third alignment.

In response to determining that the first alignment and the third alignment agrees on an adapter position, the method of determining the first consensus match score can include determining the first consensus match score based on a first sum of matched bases and a second sum of total bases from the first alignment and the third alignment as 1-P (m>=M1+M3), where m has a distribution of binomial (n1+n3, p). For example, we consider the match score as adding the matched bases and the total number of alignment bases such that if we have 3 matches out of 3 bases in the first alignment and 9 matched bases out of 10 bases, respectively the match score can be 1-P (m>=12), where m˜binomial (13, p) for a sum of 12 matches of 13 total aligned bases.

In response to determining that the first alignment and the third alignment disagrees on the adapter position, the operation of determining the first consensus match score can include: determining a first candidate match score based on a first sum of matched bases and a second sum of total bases from (1) a first adapter position obtained from the first alignment and from (2) assuming the first adapter position is valid, aligning the first adapter to the tail of the first sequencing read at the first adapter position indicated in the first alignment. Continuing referring to the example above, from (1) the first adapter position, there are 9 matched bases out of 10. From (2), the first adapter is aligned to the tail of the first sequencing read as:

R1 read:
CCTCGATCCCAGATCGGAGA
1st adapter:
AGATCGGAAG

There are 3 matched bases out of 3 bases. The first candidate match score can be determined based on 9+3 matched bases out of 10+3 bases. In this example, the first candidate match score is 1-P (m>=12), where m has a distribution as Binom (13, 0.25). The first candidate match score is 0.9999994039535522.

The operation of determining the first consensus match score can include: determining a second candidate match score based the first sum of matched bases and the second sum of total bases from (1) a second adapter position obtained from the third alignment and from (2) assuming the third alignment is valid, aligning the tail of the first sequencing read to the head of the second sequencing read at the second adapter position. Continuing referring to the example above, from (1) the second adapter position, there are 8 matched bases out of 10. From (2), the tail of the first sequencing read is aligned to the head of the second sequencing read at the second adapter position as:

R1 read:
CCTCGATCCCAGATCGGAGA
R2 head:
CACAGATCGG

So that there are 10 bases from the adapter as indicated in (1). We see 5 out of 10 bases are matched at this second adapter position. The second candidate match score can be determined based on 8+5 matched bases out of 10+10 bases. In this example, the first candidate match score is 1-P (m>=13), where m has a distribution as Binom (20, 0.25). The second candidate match score is 0.9998162959418551.

The operation of determining the first consensus match score can include selecting a score from the first and second candidate match scores as the first consensus score. The selected score can be a score that is higher. The selected score can be a score that is higher and also satisfy a predetermined threshold. Continuing to refer to the example above, the first candidate score is higher than the second candidate score, so the first consensus score is the first candidate score.

In some embodiments, the operation 520 includes generating the second consensus position using the second alignment and the fourth alignment. In some embodiments, the operation 520 comprises generating a third adapter position based on the second alignment and generating a fourth adapter position based on the fourth alignment. The third or fourth adapter position can indicate at what position the insert of the reads end and the adapter bases starts. For example, the third adapter position can be that the adapter starts at the 15^{th} base of the R1 read.

In response to determining that the third adapter position agrees with the fourth adapter position, the operation 520 includes determining the second consensus position as the third adapter position.

In response to determining that the second alignment and the fourth alignment agrees on the adapter position, the operation 520 includes determining a second consensus match score based on a third sum of matched bases and a fourth sum of total bases from the second alignment and the fourth alignment. Continuing referring to the example above, the third sum of matched bases from the second and the fourth alignment is 3+9, and the total number of bases is 10+30, so that the second consensus match score is 0.9999994039535522.

In response to determining that the second alignment and the fourth alignment agrees on the adapter position, the operation 520 includes determining a third candidate match score based on a third sum of matched bases and a fourth sum of total bases from a third adapter position obtained from (1) the second alignment and(s) from aligning the second adapter to the tail of the second sequencing read at the third adapter position, assuming the third adapter position is valid. The operation 520 can further include determining a fourth candidate match score based the third sum of matched bases and a fourth sum of total bases from a fourth adapter position obtained from (1) the fourth alignment and from (2) aligning the tail of the second sequencing read to the head of the first sequencing read at the fourth adapter position by assuming the fourth adapter position is valid. In some embodiments, the operation 520 can further include selecting a score from the third and fourth candidate match scores as the second consensus score. The selected score can be the higher score from the third and fourth candidate match scores. The selected score can also satisfy a predetermined threshold.

In some embodiments, the method 500 includes an operation of determining a trimming position based on the first consensus position, the second consensus position, a first consensus match score, and a second consensus match score. If the first consensus position and the second consensus position agree with each other, the trimming position can be determined as the agreed position. In response to determining that the first and second consensus positions are different, then the position with a higher consensus score can be selected as the trimming position. In the example as above, the first consensus position and the second consensus position indicate last 3 bases from R1 read and R2 read are from the adapter, and the consensus match scores are identical, so the trimming position is at the last 3 bases. So that the sequencing reads after trimming become:

R1 read:
CCTCGATCCCAGATCGG
R2 read:
CCGATCTGTGATCGAGG

In some embodiments, the probability of P (m>M) can be pre-calculated, where m has a binomial distribution as Binom (n,p). For every M in the range [0, n] and m in the range [0, M], P (m>M) or P (m>=M) can be precalculated and saved, e.g., in a table. The table can be used as a look-up table so that for each pair of M and n, there is a unique table element that corresponds to the pair, which is the value of P (m>M). For example, the look-up table can have a column number that corresponds to a value of M, and a row number corresponding to a value of n. Pre-calculation of the look-up table can save computational time during adapter trimming and can be conveniently reused in adapter trimming within a run or across different runs. In some embodiments, P (m>=M) is the probability of observing at least M matches out of a total number of n bases (a maximal possible number of matches) by random chance. P (m>M) is the probability of observing greater than M matches out of a total number of n bases (a maximal possible number of matches) by random chance.

In some embodiments, the computer-implemented method 500 may include an operation of performing one or more preprocessing steps before operation 510.

In some embodiments, this operation of performing one or more preprocessing steps can be performed by the FPGA(s). In some embodiments, the data after the operation can be communicated by the FPGA(s) to the CPU(s) so that CPU(s) can perform subsequent operation(s) in method 500 using such data.

The one or more preprocessing steps can comprise background subtraction. The background subtraction is configured to remove at least some background signal that may interfere with the signal of interest, i.e., image intensities of the polonies. The background signal can be noise caused by multiple sources including the flow cell 112, the imager 115, the sequencer 114, and other sources. The background subtraction can be adjusted to avoid over subtraction.

The one or more preprocessing steps can include image sharpening so that image intensities of polonies can be optimized in consideration of their surroundings in the flow cell images. For example, a Laplacian of Gaussian (LoG) filter can be used for sharpening.

The one or more preprocessing steps can include image registration so that image intensities of polonies can be registered relative to each other. For example, the image intensities can be registered to the template as disclosed herein.

The one or more preprocessing steps can include intensity offset adjustment that can remove the offset in the intensity that has not been removed during background subtraction.

The one or more preprocessing steps can include color correction to remove interference of one channel from other channels or colors.

The one or more preprocessing steps can include phasing and prephasing correction which is configured to correct image intensities within a specific cycle by removing intensity biases caused by sequencing of DNA fragments that are out of synchronization from other fragments by either falling behind or getting ahead.

The one or more preprocessing steps can include intensity normalization so that the image intensity of polonies from different channels can be normalized to be within a predetermined range.

The one or more preprocessing steps can include quality score estimation so that quality of base calling using the flow cell images acquired by the imager 116 can be estimated before actual base calling is performed.

The one or more preprocessing steps can include base calling using various base calling algorithms. After base calling, each polony can have multiple DNA fragments as multiple sequences of bases.

The one or more preprocessing steps can include saving sequencing read to the sequencing system or external data storage such as the cloud. In some embodiments, the sequencing reads can be saved in binary format directly. In some embodiments, after base calling, the sequencing reads are generated in binary format and directly saved in the same binary format. The method 500 disclosed herein can advantageously obtain the sequencing reads and directly perform adapter trimming operations disclosed herein in binary format without the additional processing to covert the binary format to a different format as existing adapter trimmers commonly do.

In some embodiments, before operation 510, the method 500 includes an operation of obtaining, by a processor herein, a plurality of sequencing reads. Such operation of obtaining can comprise actively retrieving or passively receiving the plurality of sequencing reads after base calls have been generated and recorded. The sequencing reads can be from a sequencing run that is completed or in progress. Each sequencing read may be a pair-end sequencing read.

In some embodiments, the method 500 includes an operation of trimming the first sequencing read, the second sequencing read, or both at the trimming position.

In some embodiments, the method 500 include an operation of converting the trimmed first sequencing read, the trimmed second sequencing read, or both to a predetermined format that is not binary format.

In some embodiments, the method 500 includes an operation of recording the trimmed first sequencing read, the trimmed second sequencing read, or both in the predetermined format in a data storage that is either part of the sequencing system 100 or external to the sequencing system 100. For example, the trimmed sequencing reads can be saved to a data storage device on a user's computer system 400 external to the sequencing system 100.

In some embodiments, the method 500 includes communicating the trimmed first sequencing read, the trimmed second sequencing read, or both in the predetermined format to a processing unit. In some embodiments, the trimmed sequencing reads can be communicated to a user's computer system 400. In some embodiments, the processing unit is a central processing unit (CPU). In some embodiments, the processing unit, e.g., a user's computer 400, is configured to generate a sequencing result for display to a user based on the trimmed first sequencing read, the trimmed second sequencing read, or both in the predetermined format. In some embodiments, the methods 500 includes an operation of generating or outputting a sequencing result, including multiple sequencing reads, that a user can rely on for making genetic determinations without error caused by adapter(s). The predetermined format is a format that is not binary but a format that can be displayed and more easily perceived by the user than the binary format.

FIGS. 3A-3B show simulation of adapter trimming results using the methods disclosed here and existing adapter trimmers in simulated sequencing runs. “b2f (PE)” stands for the methods disclosed herein, using 4 alignments selected from (a)-(d) in operation 510. “b2f (naïve)” stands for the “naïve” methods that uses 4 alignments selected from (a)-(d) in operation 510 with accuracy thresholds, e.g., 90% matched bases are higher than 70% matched bases, without using random matching and binomial distribution. “cutadapt” and “fastp” are two existing adapter trimming methods. Table 1 shows that the methods disclosed herein, in a simulated sequencing run with about 20 k paired end reads, has a highest sensitivity and highest phi coefficient among all 4 methods compared. The phi coefficient is a parameter that can indicate the quality of trimming that is calculated based on the confusion matrix including true positives, true negatives, false positives, and false negatives. The specificity of the methods disclosed herein is the 2nd highest. However, the computational time or runtime of the present methods are faster, which only ranges from a couple of seconds to less than 10 minutes, while “cutadapt” and “fastp” can take at least a couple of hours.

The methods disclosed herein advantageously remove the need to have additional indel processing to adapter trimming. The indel processing can be intrinsic to the methods disclosed herein. For example, if there is an indel in the adapter, the third and fourth alignments from (c) and (d) may be affected, so that the corresponding match scores may suffer. But the first and second alignments (a) and (b) is not affected and can produce high match scores thus an accurate estimate of adapter position. When there is an indel in the insert but not the adapter, the first and second alignments (a) and (b) can be affected by the indel, but the other alignments can still produce accurate estimates of adapter position. Table 2 (in FIG. 3A) shows the quality of adapter trimming when there is indel, i.e., insertion and/or deletion in the adapter and the insert, in a simulated sequencing run with about 100 k paired end reads. The methods disclosed herein has a highest sensitivity and highest phi coefficient among all 4 methods compared. The computational time or runtime of the present methods are faster, which only ranges from a couple of seconds to less than 10 minutes, while “cutadapt” and “fastp” can take at least a couple of hours.

The methods disclosed herein advantageously work with asymmetric read lengths when the indel can occur anywhere in the read(s). In a simulated sequencing run with about 100 k paired end reads, the length (or number of sequencing cycles) of the reads can vary from about 70 bases to about 180 bases. Table 3 (in FIG. 3B) shows that the methods disclosed herein has a highest sensitivity and highest phi coefficient among all 4 methods compared, the specificity is the second highest among 4 different methods.

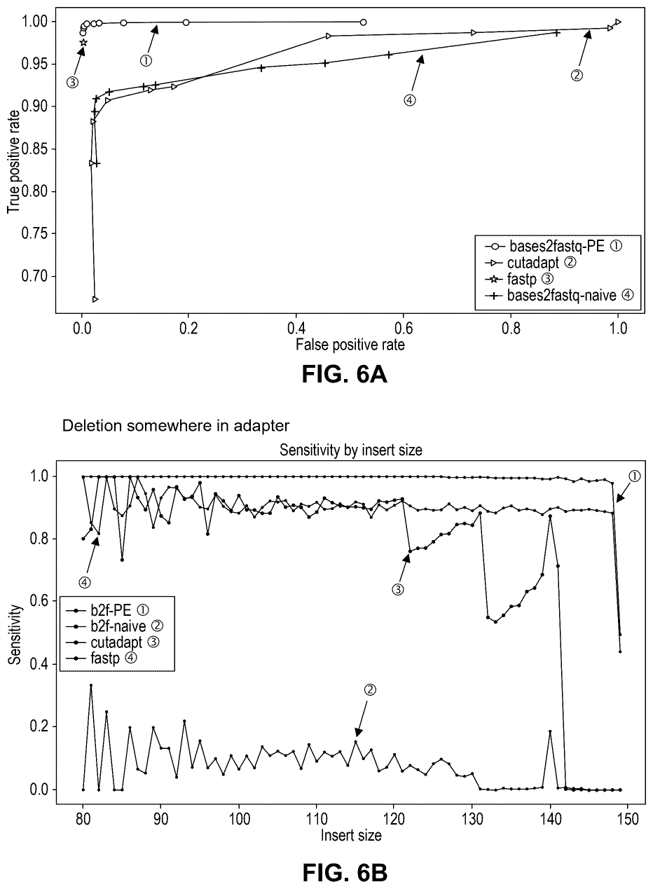

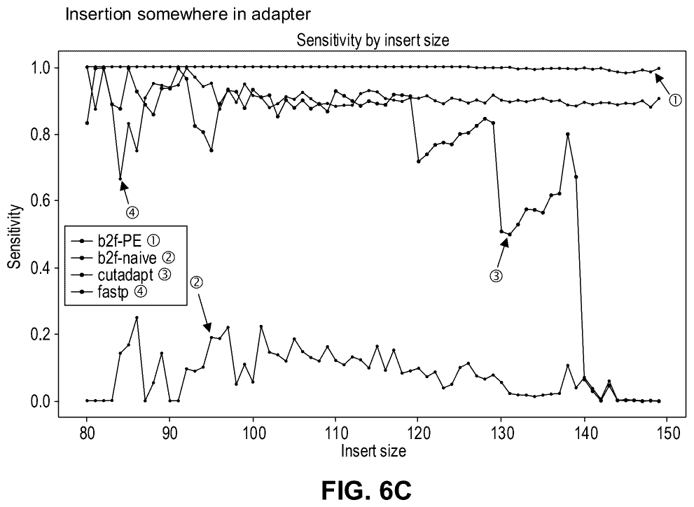

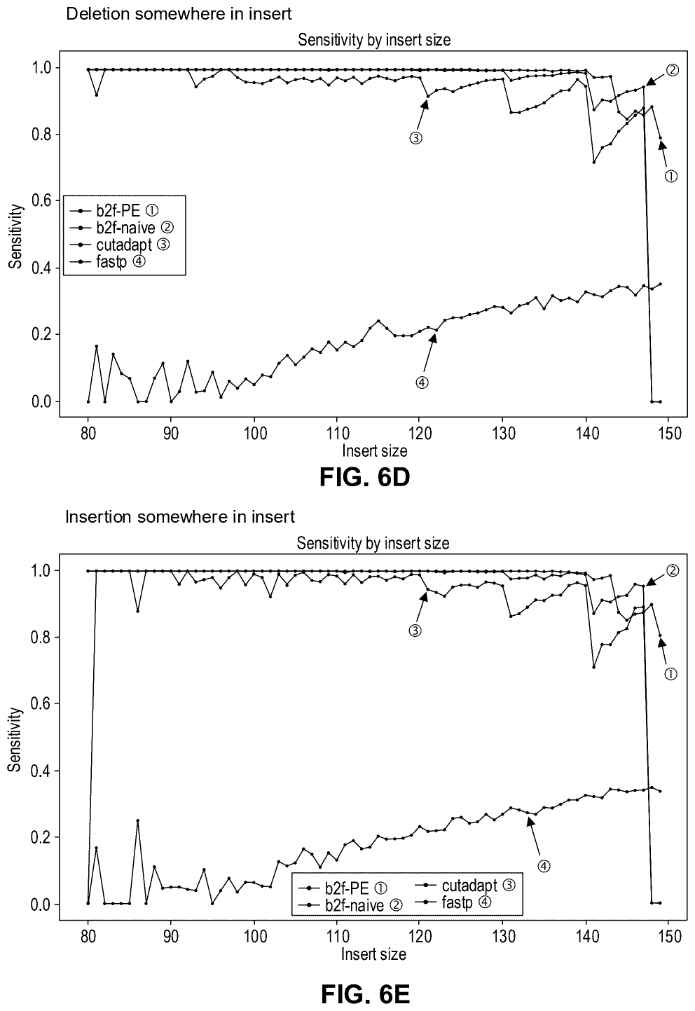

FIG. 6A show the receiver operating characteristic (ROC) curve of the methods disclosed herein in comparison to other existing methods in analyzing the same adapter trimming dataset. The ROC curve is generated by plotting the true positive rate against the false positive rate at various threshold settings. The ROC curve can show the sensitivity or recall as a function of fall-out(s). Different simulated runs are performed with different match score thresholds. The “b2f-PE” accuracy thresholds are: 0.995, 0.999, 0.9995, 0.9999, 0.99995, 0.99999, 0.999999, 0.9999999, and 0.99999999, each for a separate simulated sequencing run. The “b2f-naïve accuracy thresholds are: 0.5, 0.55, 0.6, 0.65, 0.7, 0.75, 0.8, 0.85, 0.9, and 0.95. The “cutadapt” max errors are set as: 0.5, 0.45, 0.4, 0.35, 0.3, 0.25, 0.2, 0.15, 0.1, and 0.05. No accuracy threshold is available for “fastp.” The method disclosed herein labeled as “bases2fastq-PE” has the best the ROC curve among all 4 methods, indicating better performance of the methods disclosed herein over existing methods, in its accuracy and sensitivity.

FIGS. 6B-6E shows the comparison of sensitivities among 4 different methods when there is an indel scenarios including: deletion in the adapter (FIG. 6B), insertion in the adapter (FIG. 6C), deletion in the insert (FIG. 6D), insertion in the insert (FIG. 6E) across different insert sizes in the simulated runs. The methods disclosed herein, i.e., “b2f_PE” has the highest sensitivity among all methods across the different insert sizes in different indel scenarios.

The embodiments disclosed herein focused on adapters that are attached at the tail or the 3′ end of sequencing reads. However, it is worth noting that in the embodiments where the adapters are attached to the head or 5′ end of sequencing reads, the operations can be similarly performed by correspondingly changing the third alignment and fourth alignment by aligning the adapter to the head of the sequencing reads so that the third alignment is selected from (c) aligning the first adapter to the head of the first sequencing read at one or more third positions; and the fourth alignment is selected from (d) aligning the second adapter to the head of the second sequencing read at one or more fourth positions in operation 510.

In some embodiments, the operation 510 can include selecting all four alignments, i.e., the first alignment from (a), the second alignment from (b), the third alignment from (c), and the fourth alignment from (d) as disclosed herein.

In some embodiments, the operation 510 can be simplified to include selecting less than 4 alignments, for example 3 alignments. In some embodiments, the 3 alignments can be:
the first alignment from (a), the second alignment from (b), and the third alignment from (c);
the first alignment from (a), the third alignment from (c), and the fourth alignment from (d);
the first alignment from (a), the second alignment from (b), and the fourth alignment from (d); or
the second alignment from (b), the third alignment from (c), and the fourth alignment from (d).
   In embodiments using 3 alignments, the operation 520 of generating the first consensus position or the second consensus position can be simplified if only one alignment instead of two alignments are available so that the sum of the matched bases and the sum of total bases are replaced by the matched bases and total bases from the single alignment that is available. For example, if the first consensus portion is calculated based on first and third alignment, and the third alignment is not used, then based determined from the first alignment instead of the summed bases are used. Similarly, the operation of determining the first consensus match score, and/or the second consensus match score is equivalent to determining the match score of a single alignment that is available instead of using both alignments. The other operations with 3 alignments can remain identical to the methods that use 4 alignments.

Using 2 or 3 alignments instead of 4 alignments may simplify the methods and reduce computational burden and time to perform adapter trimming operations. However, it can be more prone to error and may not offer as accurate results as using 4 alignments.

### Adapter Determinations

FIG. 5B shows a flow chart of a computer-implemented method 600 for adapter determination after base calls have been generated during sequencing analysis, according to some embodiments. The method 600 can include some or all of the operations disclosed herein. The operations may be performed in but is not limited to the order that is described herein.

The method 600 can be performed by one or more processors disclosed herein. In some embodiments, the processor can include one or more of: a processing unit, an integrated circuit, or their combinations. For example, the processing unit can include a central processing unit (CPU) and/or a graphic processing unit (GPU). The integrated circuit can include a chip such as a field-programmable gate array (FPGA). In some embodiments, the processor can include the computing system 400.

In some embodiments, some or all operations in method 600 can be performed by the FPGA(s). In embodiments when some operations are performed by FPGA(s), the data after an operation performed by the FPGA(s) can be communicated by the FPGA(s) s to the CPU(s) so that CPU(s) can perform subsequent operation(s) in method 600 using such data. Similarly, data can also be communicated from the CPU(s) to the FPGA(s) for processing by the FPGA(s). In some embodiments, all the operations in method 600 can be performed by CPU(s). Alternatively, the operations performed by CPU(s) can be performed by other processors such as the dedicated processors, or GPU(s). In some embodiments, all the operations in method 600 can be performed by FPGA(s).

In some embodiments, the methods 600 is performed after cycle N has been completed, while sequencing, image acquisition of cycle N+1 is yet to be performed. the methods 600 is performed after the entire sequence run is completed. In some embodiments, cycle N is the current cycle. N can be any non-zero integer. In some embodiments, cycle N can be determined based on the adapter lengths and/or sequence insert length. In some embodiments, cycle N is the cycle after the cycles corresponding to adapter(s). In some embodiments, cycle N can be determined if insert lengths are within a range, e.g., 100 to 150. For example, N can be any integer from 30 to 300 or 20 to 400. In some embodiments, N is not the last cycle in the sequencing run. In some embodiments, N is in the first half or first one third of the total number of sequencing cycles in the sequencing run. In some embodiments, the method 600 is performed while the sequencing run is being performed. In some embodiments, the method 600 is performed and the result of adaptor trimming or adapter determination to determine if the sequencing run in progress should be stopped or not if the adapter lengths or insert lengths are pre-known before cycle N.

The method 600 can comprise an operation 610 of obtaining, by a processor herein, a plurality of sequencing reads. The sequencing reads can be from a sequencing run that is completed or in progress. Each sequencing read may be a pair-end sequencing read. Each pair-end sequencing read may comprise a first and second sequencing read, wherein each of the first and second sequencing read comprise a sequence of nucleotide bases. FIG. 2 shows the example pair-end sequencing reads, i.e., Read 1 (R1) and Read 2 (R2).

The sequencing read can be generated by the system 100 and communicated to a processor within the system 100. Alternatively, the sequencing read can be outputted by the system 100 to a cloud or a processor, e.g., computer system 400, external to the system 100.

The sequencing reads can be generated from a pair-end sequencing run, e.g., by primary analysis. The sequencing reads may have not been demultiplexed yet. In some embodiments, the sequencing reads may have been demultiplexed. Each sequence run may include multiple number of paired sequencing reads. For example, a single run can include various number of sequencing reads, e.g., 100 to 10⁸ sequencing reads. The amount of sequencing data, e.g., 100,000 pair end sequencing reads with each read has a length in the range from about 30 bases to about 300 bases, and its processing, handling, and recording demands methods like those disclosed herein that are computationally efficient and time effective to facilitate sequencing analysis, e.g., secondary analysis.

FIG. 2 shows a schematic diagram of a pair-end sequencing read according to embodiments disclosed herein. In some embodiments, each of the first sequencing read 210, the second sequencing read 220, the head of the first sequencing read 211, the head of the second sequencing read 221, the tail of the first sequencing read 212, the tail of the second sequencing read 222, the first adapter 213, and the second adapter 223 comprises a sequence of nucleotide bases. The sequence of the bases can be an orderly sequence and each base can be one of the four different bases, i.e., A, C, G, and T/U. Each sequence read may include a base per cycle per polony or cluster. The sequence of bases can be of low or unbalanced diversity in one or more cycles so that one or more types of the bases is less than 10%, 8%, 5%, or 2% of the total number of bases of that cycle.

The sequencing read 200 may be from a pair-end sequencing run. Each run may include multiple numbers of paired sequencing reads 200. For example, a single run can include about 100 k paired end sequencing reads. The amount of sequencing data, e.g., 100k paired end sequencing reads with each read has a length in the range from about 50 bases to about 400 bases, and its processing, handling, and recording demands methods like those disclosed herein that are computationally efficient and time effective. The sequencing read 200 can include a forward read (R1) 210, and a reverse read (R2) 220. Both the forward read 210 and the reverse read 220 can include an insert having a head portion 211, 221 and a tail portion 212, 222. The reads 210, 220 can have an adapter 213, 223 ligated or otherwise attached to the tail 212, 222. The insert is the sequence of bases of interest. In some embodiments, the reads 210, 220 can have an adapter ligated or otherwise attached to the head 212, 222 (not shown). In some embodiments, the reads 210, 220 can have an adapter ligated or otherwise attached to both the head 211, 221 and the tail 212, 222 (not shown). The first adapter 213 can be a 3′ adapter and the second adapter 223 can be a 5′ adapter.

In some embodiments, the insert in reads 210, 220 can have any number of bases ranging from about 8 bases to about 400 bases. In some embodiments, the insert in reads 210, 220 can have any number of bases ranging from about 16 bases to about 400 bases. In some embodiments, the insert in reads 210, 220 can have any number of bases ranging from about 16 bases to about 300 bases. In some embodiments, the insert in reads 210, 220 can have any number of bases ranging from about 32 bases to about 200 bases. In some embodiments, the insert in reads 210, 220 can have any number of bases ranging from about 64 bases to about 150 bases. In some embodiments, the insert in the forward read 210 and the insert in the reverse read 220 can be of the same length. In some embodiments, the insert in the forward read 210 and the insert in the reverse read 220 can be of different length.

In some embodiments, the head 211, 221 and/or the tail 212, 222 can have a number of bases in the range of about 8 bases to about 200 bases. In some embodiments, the head 211, 221 and/or the tail 212, 222 can have a number of bases in the range of about 16 bases to about 150 bases. In some embodiments, the head 211, 221 and/or the tail 212, 222 can have a number of bases in the range of about 16 bases to about 80 bases. In some embodiments, the head 211, 221 and/or the tail 212, 222 can have a number of bases in the range of about 16 bases to about 64 bases. In some embodiments, the head 211, 221 and/or the tail 212, 222 have a number of bases in the range of about 16 bases to about 32 bases. In some embodiments, the head 211, 221 and/or the tail 212, 222 have at least 16 bases. In some embodiments, each of the head 211, 221 and the tail 212, 222 can be of a different number of bases. In some embodiments, two or more of the head 211, 221 and the tail 212, 222 can have a same number of bases.

In some embodiments, the first adapter 213 and/or the second adapter 223 has about 4 bases to about 100 bases. In some embodiments, the first adapter 213 and/or the second adapter 223 has about 4 bases to about 64 bases. The first adapter or the second adapter can have about 16 bases to about 64 bases. The first adapter or the second adapter can have about 16 bases to about 40 bases. In some embodiments, the first adapter 213 and/or the second adapter 223 has about 30, about 31, about 32, about 33, or about 34 bases. In some embodiments, the first adapter 213 and/or the second adapter 223 has 30, 31, 32, 33, or 34 bases. The first adapter 213 and/or the second adapter 223 can have at least 16 bases. The first adapter 213 and the second adapter 223 can have a same number of bases. In some embodiments, the first adapter 213 and the second adapter 223 can have different numbers of bases. For example, the first adapter can have more bases than the second adapter 223.

Exemplary first and second sequencing reads are shown as R1 read and R2 read in relation to method 500 herein.

In some embodiments, the method 600 can include an operation 620 of selecting one or more alignments from:
a first alignment from (a) aligning a tail of a first sequencing read 212 to a head of a second sequencing read 221 at one or more first positions; and
a second alignment from (b) aligning the tail of the second sequencing read 222 to the head of the first sequencing read 211 at one or more second positions.

In some embodiments, operation 620 can include selecting both alignments, i.e., the first alignment from (a) and the second alignment from (b).

In some embodiments, selecting a first alignment from (a) aligning a tail of a first sequencing read 212 to a head of a second sequencing read 221 at one or more first positions comprises: an operation of obtaining the reverse complement bases of the head of the second sequencing read. Subsequently, selecting a first alignment from (a) can include an operation of aligning the reverse complement of the head of the second sequencing read to the tail of the first sequencing read at a position, and calculating a match score at that position, and then moving the reverse complement bases of the head of the second sequencing read relative to the tail of the first sequencing read to a next position, by a base, and repeating the calculation of the match score at the next position. Such moving of the two sequences relative to each other and calculation of match scores can be repeated to cover all possible different positions from aligning a single base on one end to aligning all possible bases of the tail of the first sequencing read to the head of the second sequencing read, and then to aligning a single base again on the other end of the sequence of nucleotide bases. Subsequently, the first alignment can be selected based on the match scores at the one or more first alignment positions.

In some embodiments, the operation 620 comprises determining, by the processor, the adapter position based on a plurality of match scores, each match score calculated based on a first number of matched bases and a second number of total bases at the one or more first positions and the one or more second positions. The adapter position may be a nucleotide base position where the adapter starts and the insert portion of the sequence read ends. The adapter position is estimated using operation 620. In some embodiments, the operation 620 comprises determining, by the processor, the adapter position based on a maximum match score among the plurality of match scores. In some embodiments, the operation 620 comprises determining, by the processor, the adapter position based one or more match scores that meet a predetermined threshold among the plurality of match scores.

For example, in a sequencing read with 2×20 bases, R1 is CCTCGATCCCAGATCGGAGA, and R2 is CCGATCTGTGATCGAGGAGA, which is the reverse compliment of R1 with 1 sequencing error. The last 3 bases of R1 and R2 as bases from the adapter(s). In this embodiment, the head of R2 read is the reverse complement of the first 10 bases of R2. So, R2 head is CACAGATCGG. The determined R2 head is then aligned with the tail of R1, and moves base by base toward the head of R1 (moves from right to left) at different alignment positions with 1 base aligned, 2 bases aligned, etc. When there are 10 bases aligned, there are multiple alignment positions as the R2 head can keep moving left so that it aligns with all different options of 10 bases in R1. A match score is calculated at each different alignment position as R2 head keeps moving base by base. The first alignment is determined when R2 head aligns with the 8^{th} base to the 17^{th} base in R1 as:

R1 read:
CCTCGATCCCAGATCGGAGA
R2 head:
CACAGATCGG

The first alignment is determined based on the match scores at all possible alignment positions. There are a total number 10 aligned bases, the maximum matched score is 9 out of 10 are matched bases, so that n=10, M=9.

The match score is based on a first number of matched bases at the first alignment and a second number of total aligned bases. In this example, there are 9 matched bases out of a total number of 10 aligned bases. In some embodiments, the match score is based on a probability that the first number of matched bases does not occur randomly. In some embodiments, the match score is based on a probability that the first number of matched bases over a total number of aligned bases does not occur randomly.

In some embodiments, the match score herein is calculated similarly using the methods disclosed in relation to method 500.

Similarly, as the first alignment, the second alignment can be determined. R1 head can be similarly defined as the reverse complement of the first 10 bases of R1. In this particular example, R1 head is GGGATCGAGG. The second alignment with highest match score is aligning R1 head from 8^{th} to 17^{th} bases of R2 read, as:

R2 read:
CCGATCTGTGATCGAGGAGA
R1 head:
GGGATCGAGG

Based on the first alignment with the highest score, and the second alignment with the highest match score, the adapter position is from 18^{th} to 20^{th} bases of the R1 read and the R2 read. As such, the insert length determined from both first and second alignments agrees and it is 17 bases.

The method 600 may include an operation 630 of obtaining, by the processor, a plurality of adapter sequences from the plurality of pair-end sequencing reads based on the determined adapter position in operation 620. The adapter sequences starts with 3 bases of AGA for both 3′ and 5′ adapter sequences at the 18^{th} base of the R1 and R2 reads. The adapter sequence can be whatever sequence of bases that gets trimmed at the determined adapter position in operation 620.

In some embodiments, the first and second sequence read comprise only one adapter, e.g., either 3′ or 5′ adapter. In some embodiments, the first and second sequence read comprise two or more adapters that are different. In some embodiments, the first and second sequence read comprise two or more adapters that are identical. In some embodiments, the adapter herein comprises 4 to 96 bases. In some embodiments, the adapter comprises 6 to 48 bases.

In some embodiments, the method 600 include an operation 640 of repetitively performing one or more operations to determine what the adapter sequences obtained from operation 630 are until a stopping criterion is met. The operation 640 may include: an operation 641 of selecting a seed adapter sequence from the plurality of adapter sequences; an operation 642 of determining one or more adapter sequences among the plurality of adapter sequences that satisfy a similarity threshold in comparison with the seed adapter sequence; operation 643 of determining, for each base position, a base of A, G, C, or T/U based on a corresponding count of different bases at the corresponding base position of the one or more determined adapter sequence and a predetermined threshold, thereby generating an individual candidate adapter; and operation 644 of removing the seed adapter sequence and the one or more determined adapter sequences from the plurality of adapter sequences.

In some embodiments, the method 600 may further comprise an operation of forwarding, by the processor, the individual candidate adapters to a memory device, a display, or both.

The stopping criterion can be customized by a user. For example, the stopping criterion can be that all the adapter sequences have been through operations 641 to 644 at least once and there is no other sequences left from the pool of adapter sequences obtained in operation 630.

The similarity of two sequences may be determined using various methods. For example, the similar threshold is determined by one or more of: a Hamming distance, a Smith-Waterman algorithm, and a Needleman-Wunsch algorithm. In embodiments where similarity is determined by the Hamming distance, a Hamming distance of 3 or 4 may be the similarity threshold for adapter sequences of 30 to 40 bases.

The method 600 may further comprise an operation 643 of determining, for one or more base positions, a base of A, G, C, or T/U based on a corresponding count of different bases at the corresponding base position of the one or more determined adapter sequence and the predetermined threshold.

In response to determining that n base positions out of a total number of positions fail to satisfy the predetermined threshold, removing the seed adapter sequence and the one or more determined adapter sequences from the plurality of adapter sequences without generating a candidate adapter.

In some embodiments, the predetermined threshold comprises a concordance of greater than 50%, 55%, 60%, or more among a total number of bases at the corresponding base position. In some embodiments, the predetermined threshold further includes that the one or more adapter sequences comprises at least 30, 40, 50, 60, 80, 100, or more sequences. In some embodiments, the predetermined threshold further includes that the one or more adapter sequences comprises at least 30, 40, 50, 60, 80, 100, or more counts of the same base in a particular base position. For example, at the first base position, there are 55 observations of A over 60 total observations, and 5 observation of other three types of bases, the predetermined threshold is 50 counts of the same bases is met, and the first base position is A. As another example, there are 100 observations of G in the first base position out of 300 total observations, the method 600 cannot determine the first base as G because the concordance of greater than 50% has not been satisfied. When there are more than the predetermined number of base positions, e.g., 5, 6, or more base positions of the adapter that cannot be determined, the seed adapter sequence and the one or more determined adapter sequences may be removed from the plurality of adapter sequences without generating a candidate adapter. When there are less than the predetermined number of base positions, e.g., only 1 or 2 base positions of the adapter that cannot be determined, the seed adapter sequence and the one or more determined adapter sequences may be removed from the plurality of adapter sequences and a candidate adapter sequence can still be generated with the 1 or 2 undetermined base positions. The predetermined number of undetermined bases can be 1, 2, 3, 4, 5, or more. The predetermined number of undetermined bases can be a percentage of undetermined bases. For example, the percentage of undetermined bases is less than 20%, 15%, 10%, 5%, 2%, or 1% of the total number of bases. For example, 2 undetermined bases among 20 total bases would be 10% undetermined percentage of bases.

In some embodiments, the method 600 further comprises selecting a second seed adapter sequence from the plurality of adapter sequences; and determining one or more second adapter sequences among the plurality of adapter sequences that satisfy a similarity threshold in comparison with the second seed adapter sequence, and subsequently repeat performing operations 643-644 for the second seed adapter sequence.

In some embodiments, the method 600 includes an operation of selecting a second seed adapter sequence from the plurality of adapter sequences; in response to determining that no adapter sequences among the plurality of adapter sequences that satisfy a similarity threshold in comparison with the second seed adapter sequence, removing the second seed adapter sequence from the plurality of adapter sequences without generating a candidate adapter.

### Exemplary Adapter Determination Repeating Operations 641-644

In this particular embodiment, we consider a sequencing run with the following two adapter sequences: AAAA and TTTT.

Using operations 610-630, we find the following adapter sequences: 1) AAAG; 2) AATA; 3) AAAA; 4) TGCT; 5) TTTT; 6) TATT; 7) TTTT; 8) AAAA; 9) AAAA; 10) AAAA; 11) TTTT; 12) CTTT; 13) ACAG; and 14) TGTA. In the first repetition of operations 641-644, the first sequence AAAG is treated as a seed sequence. The first seed sequence can be selected randomly from the pool of adapter sequences. Using a similarity metrics, e.g., Hamming distance, to go through all the other sequences in the pool, the following 6 sequences match the seed sequence satisfying the similarity threshold: AAAG; AATA; AAAA; AAAA; AAAA; and AAAA. For each one that satisfies the similarity threshold in comparison with the seed sequence (e.g., within hamming distance 1 of the seed sequence), determining a base count at each base position for each of the 4 different types of bases. The base count is determined using a base frequency matrix in this embodiment, which is [number of base position by different bases] (in this case 4 by 4), where bases are ordered as C, A, G, T. For the 6 sequences similar to the seed, the matrix is: [0,6,0,0; 0,6,0,0; 0,5,0, 1; 0,5,1,0]. The first row “0,6,0,0” implies that for the first base position, there are 0 C's, 6 A's, 0 G's and 0 T's. For each base position or each row, we will determine the base when there are at least 4 observations and at least 75% concordance. So the detected adapter is AAAA.

After the determination of the first adapter sequence, we remove all of the sequences that matched the seed AAAG and the seed sequence from the pool of sequences. After the removal, the sequences now include: TTCT; TTTT; TATT; TTTT; TTTT; CTTT; ACAG; and TGTA.

Repeating operations 641-644, TTCT is the next seed sequence. The sequences that meet the similarity threshold include: TTCT; TTTT; TATT; TTTT; TTTT; and CTTT.

The base count for each base position is: [1,0,0,5; 0,1,0,5; 1,0,0,5; 0,0,0,6]. Using the same predetermined threshold of at least 4 occurrence and 75% concordance, the detected adapter is TTTT.

After removing all the sequences that matched the previous seed, the list of observations is down to two: ACAG; and TGTA. Setting ACAG as the next seed sequence. No other sequence match ACAG that satisfy the similarity threshold, so ACAG is remove from the list. And a new repetition starts. TGTA is now set as the seed sequence. But there are no more sequences in the pool, so TGTA can also be removed from the pool. And the operation 640 is completed with a stopping criterion that no more sequences is left in the pool that has not gone through 641-644. There are two different sequences detected: AAAA and TTTT. The two different sequences can be forwarded to the user and/or used for adapter trimming of sequencing reads.

In some embodiments, the method 600 further comprises an operation of: trimming, by the processor, the individual candidate adapters from the plurality of pair-end sequencing reads, thereby generating a plurality of trimmed sequencing reads. In some embodiments, the trimming operation can be accurate and reliable since the candidate adapter sequence has been determined, and trimming may be performed only when finding an exact match at the trimming position with one of the candidate adapter sequences that has been determined in operation 640. In some embodiments, trimming operation may be performed with an optional error tolerance. The optional error tolerance rate can be customized based on the adapter sequence length, sequencing kits, or various other factors. For example, the optional error tolerance rate can be 1 mismatched base, 1 deletion, 1 insertion, or a combination thereof.

In some embodiments, the method 600 further comprises: performing, by the processor, secondary analysis on the plurality of trimmed sequencing reads. In some embodiment, the method 600 further comprises: forwarding, by the processor, the plurality of trimmed sequencing reads to a remote processor for secondary analysis. In some embodiment, the method 600 further comprises: forwarding, by the processor, the plurality of trimmed sequencing reads to local FPGA within the system 100 for secondary analysis.

In some embodiments, the method 600 further comprises: trimming, the first sequencing read, the second sequencing read, or both at the trimming position; converting, by the processor, the trimmed first sequencing read, the trimmed second sequencing read, or both to a predetermined format; and recording, by the processor, the trimmed first sequencing read, the trimmed second sequencing read, or both in the predetermined format. In some embodiments, the method 600 further comprises: communicating, by the processor, the trimmed first sequencing read, the trimmed second sequencing read, or both in the predetermined format to a processing unit. The processing unit can be within the system 100 or remote from the system.

In some embodiments, the method 600, before operation 610, further comprises: generating, by the processor, the first sequencing read, the second sequencing read, or both by performing one or more primary analysis steps on flow cell images. The one or more primary analysis steps on flow cell images comprises: background subtraction; image sharpening; intensity offset adjustment; color correction; intensity normalization; phasing and prephasing correction; image registration; intensity normalization; quality score estimation; or a combination thereof.

In some embodiments, to reduce computational complexity and/or improve computational speed, each base in one or more of: the head of the first sequencing read; the head of the second sequencing read; the tail of the first sequencing read; the tail of the second sequencing read; the first adapter; and the second adapter is expressed as an integer, a bitwise integer, a binary number with a fixed number bits, e.g., 4 or 8 bits.

In some embodiments, the methods 500, 600 herein may include an operation of providing a plurality of nucleic acid template molecules immobilized on a support, e.g., a flow cell. Each nucleic acid template molecule may comprise an insert sequence of interest. The insert sequence can be different in different template molecules. Each template molecule may correspond to a polony of optical signals in flow cell images.

In some embodiments, the methods 500, 600 herein may include an operation of generating the flow cell images by conducting one or more cycles of sequencing reactions of the plurality of nucleic acid template molecules immobilized on the support. The flow cell images can be generated or acquired by the sequencing system disclosed herein. Conducting the one or more cycles of the sequencing reactions may comprise: contacting the plurality of nucleotide acid template molecules using a plurality of nucleotide reagents comprising a mixture of different types of nucleotide bases A, G, C and T/U. Individual nucleotide reagent may comprise a different detectable color label that corresponds with each different type of nucleotide base.

In some embodiments, conducting the one or more cycles of the sequencing reactions may comprise: contacting the plurality of nucleotide acid template molecules with a plurality of sequencing primers, a plurality of polymerases and a mixture of different types of avidites. An individual avidite in the mixture may comprise a core attached with multiple nucleotide arms and each arm of the individual avidite comprises the same type of nucleotide base. In some embodiments, conducting the one or more cycles of the sequencing reactions comprises: in each of the one or more cycles, imaging optical color signals emitted from nucleotide reagents that are bound to the plurality of template molecules. Imaging the optical signals may be performed by an optical system, e.g., the imager 116, disclosed herein. In some embodiments conducting the one or more cycles of the sequencing reactions may comprise: in each of the one or more cycles, acquiring the flow cell images comprising optical color signals emitted from nucleotide reagents that are bound to the plurality of template molecules.

In some embodiments, the flow cell images comprises optical signals emitted from nucleotide reagents bound to a unbalanced diversity of nucleotide bases of A, G, C and T/U among the plurality of nucleic acid template molecules immobilized on the support in the one or more cycles. In some embodiments, the plurality of polonies comprise a unbalanced diversity of nucleotide bases of A, G, C and T/U, and wherein the unbalanced diversity comprises a percentage of: (1) a number of one or more types of nucleotide bases to (2) a total number of nucleotide bases, and the percentage is less than 20%, 15%, 10%, or 5% in the cycle N.

In some embodiments, the methods 500, 600 may comprise providing a cellular sample having a plurality of concatemer molecules immobilized on a support, wherein each concatemer molecule corresponds to a target RNA of a cellular sample.

In some embodiments, the methods 500, 600 may comprise generating, by a sequencing system, flow cell images by conducting one or more cycles of sequencing reactions of the plurality of concatemer molecules immobilized on the support. Conducting the one or more cycles of the sequencing reactions may comprise: contacting the plurality of concatemer molecules using a plurality of nucleotide reagents comprising a mixture of different types of nucleotide bases A, G, C and T/U. Conducting the one or more cycles of the sequencing reactions may comprise: contacting the plurality of concatemer molecules with a plurality of sequencing primers, a plurality of polymerases, and a mixture of different types of avidites. The individual avidite in the mixture comprises a core attached with multiple nucleotide arms and each arm of the individual avidite comprises the same type of nucleotide base. Conducting the one or more cycles of the sequencing reactions may comprise: in each of the one or more cycles, imaging, by the optical system, optical color signals emitted from nucleotide reagents that are bound to the plurality of concatemer molecules. In some embodiments, conducting the one or more cycles of the sequencing reactions may comprise: in each of the one or more cycles, acquiring, by an optical system, the flow cell images comprising optical color signals emitted from nucleotide reagents that are bound to the plurality of concatemer molecules.

### Computer Systems

Various embodiments of the methods may be implemented, for example, using one or more computer systems, such as computer system 400 shown in FIG. 4. One or more computer systems 400 may be used, for example, to implement any of the embodiments discussed herein, as well as combinations and sub-combinations thereof.

Computer system 400 may include one or more hardware processors 404. The hardware processor 404 can be central processing unit (CPU), graphic processing units (GPU), or their combination. Processor 404 may be connected to a bus or communication infrastructure 406.

Computer system 400 may also include user input/output device(s) 403, such as monitors, keyboards, pointing devices, etc., which may communicate with communication infrastructure 406 through user input/output interface(s) 402. The user input/output devices 403 may be coupled to the user interface 124 in FIG. 1.

One or more of processors 404 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, and/or producing results of other proof-of-work computations for some blockchain-based applications, for example. With capabilities of general-purpose computing on graphics processing units (GPGPU), the GPU may be particularly useful in at least the image recognition and machine learning embodiments described herein.

Additionally, one or more of processors 404 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors and/or other logic to facilitate such acceleration.

Computer system 400 may also include a data storage device such as a main or primary memory 408, e.g., random access memory (RAM). Main memory 408 may include one or more levels of cache. Main memory 408 may have stored therein control logic (i.e., computer software) and/or data.

Computer system 400 may also include one or more secondary data storage devices or secondary memory 410. Secondary memory 410 may include, for example, a main storage drive 412 and/or a removable storage device or drive 414. Main storage drive 412 may be a hard disk drive or solid-state drive, for example. Removable storage drive 414 may be a floppy disk drive, a magnetic tape drive, a compact disk drive, an optical storage device, tape backup device, and/or any other storage device/drive.

Removable storage drive 414 may interact with a removable storage unit 418.

Removable storage unit 418 may include a computer usable or readable storage device having stored thereon computer software and/or data. The software can include control logic. The software may include instructions executable by the hardware processor(s) 404. These instructions, when executed by hardware processor(s) 404, may cause hardware processor(s) 404 to perform operations in accordance with the instructions. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities, or other approaches for allowing computer programs and/or other instructions and/or data to be accessed by computer system 400. Such means, devices, components, instrumentalities, or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communication path 426, which may be wired and/or wireless (or a combination thereof), and which may include any combination of LANs, WANs, the Internet, etc. Control logic and/or data may be transmitted to and from computer system 400 via communication path 426. In some embodiments, communication path 426 is the connection to the cloud 130, as depicted in FIG. 1. The external devices, etc. referred to by reference number 428 may be devices, networks, entities, etc. in the cloud 130.

Computer system 400 may also be any of a personal digital assistant (PDA), desktop workstation, laptop or notebook computer, netbook, tablet, smart phone, smart watch or other wearable, appliance, part of the Internet of Things (IoT), and/or embedded system, to name a few non-limiting examples, or any combination thereof.

It should be appreciated that the framework described herein may be implemented as a method, process, apparatus, system, or article of manufacture such as a non-transitory computer-readable medium or device. For illustration purposes, the present framework may be described in the context of distributed ledgers being publicly available, or at least available to untrusted third parties. One example of a modern use case is with blockchain-based systems. It should be appreciated, however, that the present framework may also be applied in other settings where sensitive or confidential information may need to pass by or through hands of untrusted third parties, and that this technology is in no way limited to distributed ledgers or blockchain uses.

Computer system 400 may be a client or server, accessing or hosting any applications and/or data through any delivery paradigm, including but not limited to remote or distributed cloud computing solutions; local or on-premises software (e.g., “on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); and/or a hybrid model including any combination of the foregoing examples or other services or delivery paradigms.

Any applicable data structures, file formats, and schemas may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

Any pertinent data, files, and/or databases may be stored, retrieved, accessed, and/or transmitted in human-readable formats such as numeric, textual, graphic, or multimedia formats, further including various types of markup language, among other possible formats. Alternatively, or in combination with the above formats, the data, files, and/or databases may be stored, retrieved, accessed, and/or transmitted in binary, encoded, compressed, and/or encrypted formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform and/or unique identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C#, Objective-C, Java, Scala, Clojure, Elixir, Swift, Go, Perl, PHP, Python, Ruby, JavaScript, WebAssembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 4. In particular, embodiments may operate with software, hardware, and/or operating system implementations other than those described herein.

### Optical Systems

The imager 116 in FIG. 1 can include one or more optical systems. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being images.

In some instances, improvements in imaging performance, e.g., for dual-side (flow cell) imaging applications comprising the use of thick flow cell walls (e.g., wall (or coverslip) thickness >700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Exemplary embodiments disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 μm, at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some embodiments, the numerical aperture may be at least 0.75. In some embodiments, the numerical aperture is at least 1.0. In some embodiments, the working distance is at least 850 μm. In some embodiments, the working distance is at least 1,000 μm. In some embodiments, the field-of-view may have an area of at least 2.5 mm². In some embodiments, the field-of-view may have an area of at least 3 mm². In some embodiments, the spatial sampling frequency may be at least 2.5 times the optical resolution of the fluorescence imaging system. In some embodiments, the spatial sampling frequency may be at least 3 times the optical resolution of the fluorescence imaging system. In some embodiments, the system may further comprise an X-Y-Z translation stage such that the system is configured to acquire a series of two or more fluorescence images in an automated fashion, wherein each image of the series is or can be acquired for a different field-of-view. In some embodiments, a position of the sample plane may be simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some embodiments, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm or greater. In some embodiments, the specified error threshold is 50 nm or less. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also discloser herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens may be a commercially-available microscope objective. In some embodiments, the commercially-available microscope objective may have a numerical aperture of at least 0.3. In some embodiments, the objective lens may have a working distance of at least 700 μm. In some embodiments, the objective lens may be corrected to compensate for a cover slip thickness (or flow cell wall thickness) of 0.17 mm or of greater or lesser thickness than 0.17 mm. In some embodiments, the optical system may be corrected to compensate for cover slip thickness, flow cell thickness, or distance between desired focal planes. In some embodiments, said correction may be made by inserting a corrective optic, such as a lens or optical assembly into the light path of the optical system. In some embodiments, said correction may be made without inserting a corrective optic, such as a lens or optical assembly into the light path of the optical system. In some embodiments, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some embodiments, the at least one tube lens may be a compound lens comprising three or more optical components. In some embodiments, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side imaging compared to that for a conventional system comprising an objective lens, a motion-actuated compensator, and an image sensor. In some embodiments, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm². In some embodiments, the light delivered to the specified field-of-illumination is of uniform intensity across a specified field-of-view for an imaging system used to acquire images of the support surface. In some embodiments, the specified field-of-view has an area of at least 2 mm². In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

Imaging modules and systems: It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectic focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

Embodiments of the present disclosure provide methods for sequencing immobilized or non-immobilized template molecules. The methods can be operated in system 100, for example, in sequencer 114. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. In some embodiments, the sequencing reactions employ phosphate chain labeled nucleotides.

### Library Molecules

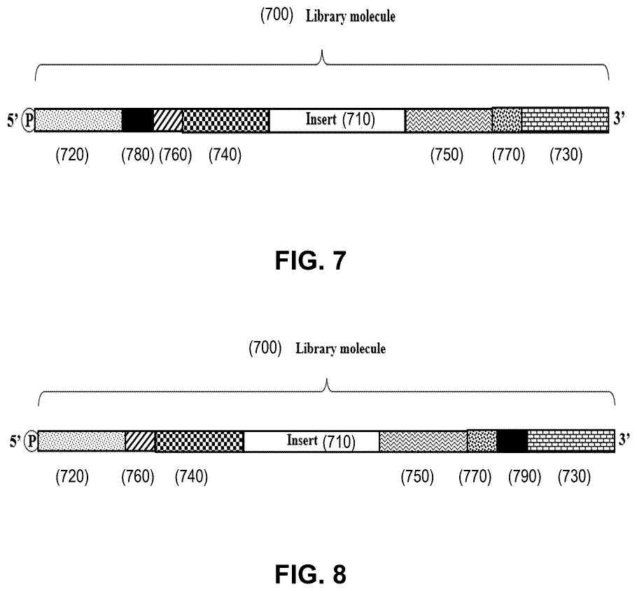

In some embodiments, the immobilized concatemers each comprise tandem repeat units of the sequence-of-interest (e.g., insert region) and any adapter sequences. For example, the tandem repeat unit comprises: (i) a left universal adapter sequence having a binding sequence for a first surface primer (720) (e.g., surface pinning primer), (ii) a left universal adapter sequence having a binding sequence for a first sequencing primer (740) (e.g., forward sequencing primer), (iii) a sequence-of-interest (710), (iv) a right universal adapter sequence having a binding sequence for a second sequencing primer (750) (e.g., reverse sequencing primer), (v) a right universal adapter sequence having a binding sequence for a second surface primer (730) (e.g., surface capture primer), and (vii) a left sample index sequence (760) and/or a right sample index sequence (770). In some embodiments, the tandem repeat unit further comprises a left unique identification sequence (780) and/or a right unique identification sequence (790). In some embodiments, the tandem repeat unit further comprises at least one binding sequence for a compaction oligonucleotide. In some embodiments, FIGS. 7 and 8 show linear library molecules or a unit of a concatemer molecule.

FIG. 7 shows an example linear single stranded library molecule (700) which comprises: a surface pinning primer binding site (720); an optional left unique identification sequence (780); a left index sequence (760); a forward sequencing primer binding site (740); an insert region having a sequence of interest (710); reverse sequencing primer binding site (750); a right index sequence (770); and a surface capture primer binding site (730).

FIG. 8 shows an example linear single stranded library molecule (700) which comprises: a surface pinning primer binding site (720); a left index sequence (760); a forward sequencing primer binding site (740); an insert region having a sequence of interest (710); a reverse sequencing primer binding site (750); a right index sequence (770); an optional right unique identification sequence (790); and a surface capture primer binding site (730).

The immobilized concatemer can self-collapse into a compact nucleic acid nanoball. Inclusion of one or more compaction oligonucleotides during the RCA reaction can further compact the size and/or shape of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yields an increased signal intensity for each concatemer. Multiple portions of a given concatemer can be simultaneously sequenced. Furthermore, a plurality of binding complexes can form along a particular concatemer molecule, each binding complex comprising a sequencing polymerase bound to a template/primer duplex and bound to a multivalent molecule, wherein the plurality of binding complexes remain stable without dissociation resulting in increased persistence time which increases signal intensity and reduces imaging time.

### Methods for Sequencing Using Nucleotide Analogs

Embodiments of the present disclosure provides methods for sequencing any of the immobilized template molecules described herein, the methods comprising step (a): contacting a sequencing polymerase to (i) a nucleic acid template molecule and (ii) a nucleic acid sequencing primer, wherein the contacting is conducted under a condition suitable to bind the sequencing polymerase to the nucleic acid template molecule which is hybridized to the nucleic acid primer, wherein the nucleic acid template molecule hybridized to the nucleic acid primer forms the nucleic acid duplex. In some embodiments, the sequencing polymerase comprises a recombinant mutant sequencing polymerase that can bind and incorporate nucleotide analogs.

In some embodiments, in the methods for sequencing template molecules, the sequencing primer comprises a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemers). In some embodiments, the plurality of nucleic acid template molecules comprise the same target sequence of interest or different target sequences of interest. In some embodiments, the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., fluorophore) that emits a detectable signal. The detectable reporter moiety comprises a fluorophore. In some embodiments, the fluorophore is attached to the nucleo-base. In some embodiments, the fluorophore is attached to the nucleo-base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleo-base. When the incorporated chain terminating nucleotide is detectably labeled, step (b) further comprises detecting the emitted signal from the incorporated chain terminating nucleotide. In some embodiments, step (b) further comprises identifying the nucleo-based of the incorporated chain terminating nucleotide.

In some embodiments, the methods for sequencing further comprise step (c): removing the chain terminating moiety from the incorporated chain terminating nucleotide to generate an extendible 3′OH group. In some embodiments, step (c) further comprises removing the detectable label from the incorporated chain terminating nucleotide. In some embodiments, the sequencing polymerase remains bound to the template molecule which is hybridized to the sequencing primer which is extended by one nucleo-base.

In some embodiments, the methods for sequencing further comprise step (d): repeating steps (b) and (c) at least once.

### Two-Stage Methods for Nucleic Acid Sequencing

Embodiments of the present disclosure provide a two-stage method for sequencing any of the immobilized template molecules described herein. In some embodiments, the first stage generally comprises binding multivalent molecules to complexed polymerases to form multivalent-complexed polymerases, and detecting the multivalent-complexed polymerases.

In some embodiments, the first stage comprises step (a): contacting a plurality of a first sequencing polymerase to (i) a plurality of nucleic acid template molecules and (ii) a plurality of nucleic acid sequencing primers, wherein the contacting is conducted under a condition suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex wherein the nucleic acid duplex comprises a nucleic acid template molecule hybridized to a nucleic acid primer. In some embodiments, the first polymerase comprises a recombinant mutant sequencing polymerase.

In some embodiments, in the methods for sequencing template molecules, the sequencing primer comprises an oligonucleotide having a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemers). In some embodiments, the nucleic acid template molecules in the plurality of nucleic acid template molecules comprise the same target sequence of interest or different target sequences of interest. In some embodiments, the plurality of nucleic acid template molecules and/or the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

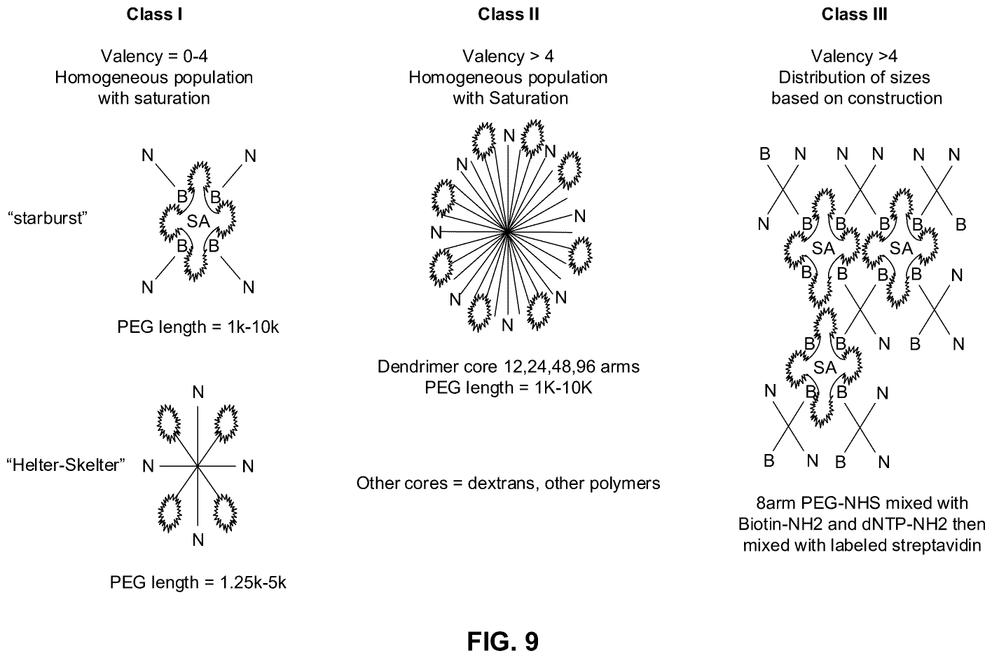

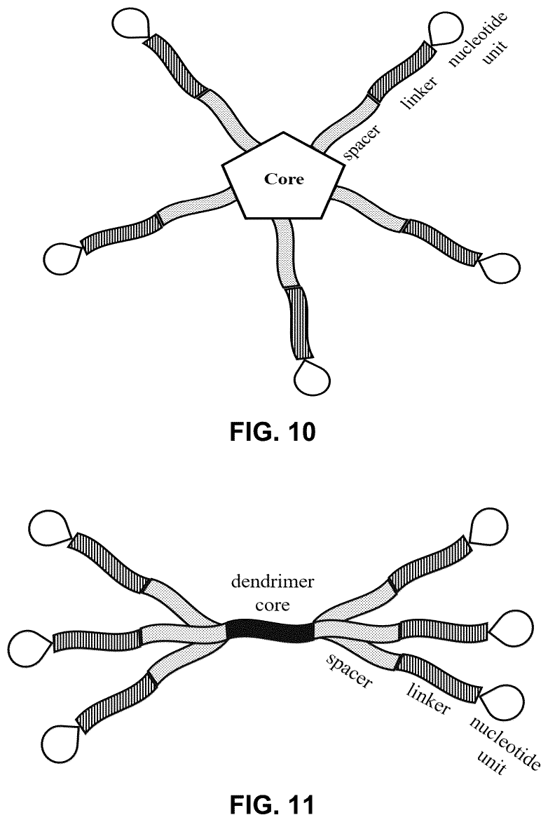

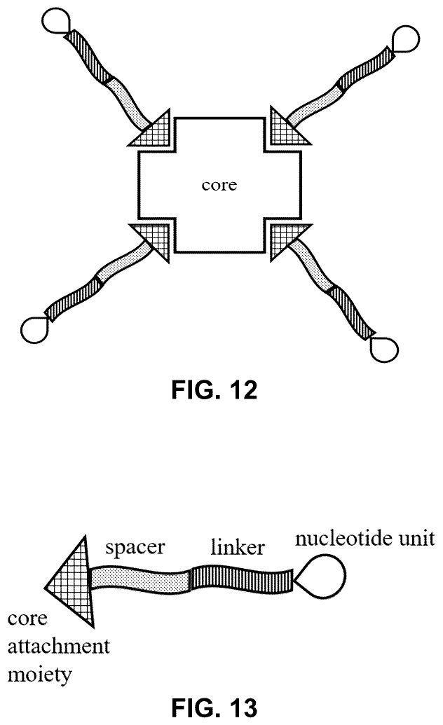

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., nucleotide unit) (e.g., FIGS. 9-13). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprise at least one multivalent molecule having multiple nucleotide arms (e.g., FIGS. 9-12) each attached with a nucleotide analog (e.g., nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety that emits a signal. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the signals emitted by the multivalent molecules that are bound to the complexed polymerases, where the complementary nucleotide units of the multivalent molecules are bound to the primers but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing further comprise step (d): identifying the nucleo-base of the complementary nucleotide units that are bound to the plurality of first complexed polymerases, thereby determining the sequence of the template molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some embodiments, the second stage of the two-stage sequencing method generally comprises nucleotide incorporation. In some embodiments, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) have an amino acid sequence that is 100% identical to the amino acid sequence as the plurality of the second sequencing polymerases of step (f). In some embodiments, the plurality of first sequencing polymerases of step (a) have an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases thereby extending the sequencing primer by one nucleo-base. In some embodiments, the incorporating the nucleotide into the 3′ end of the sequencing primer in step (g) comprises a primer extension reaction. In some embodiments, the contacting of step (g) is conducted in the presence of at least one catalytic cation comprising magnesium and/or manganese. In some embodiments, the plurality of nucleotides comprise native nucleotides (e.g., non-analog nucleotides) or nucleotide analogs. In some embodiments, the plurality of nucleotides comprise a 2′ and/or 3′ chain terminating moiety which is removable or is not removable. In some embodiments, at least one of the nucleotides in the plurality is not labeled with a detectable reporter moiety. In some embodiments, the plurality of nucleotides are non-labeled. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, when the plurality of nucleotides in step (g) are detectably labeled, the methods for sequencing further comprise step (h): detecting the complementary nucleotides which are incorporated into the primers of the nucleotide-complexed polymerases. In some embodiments, the plurality of nucleotides are labeled with a detectable reporter moiety to permit detection. In some embodiments, when the plurality of nucleotides in step (g) are non-labeled, the detecting of step (h) is omitted.

In some embodiments, when the plurality of nucleotides in step (g) are detectably labeled, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid template molecules. In some embodiments, when the plurality of nucleotides in step (g) are non-labeled, the identifying of step (i) is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once. In some embodiments, the sequence of the nucleic acid template molecules can be determined by detecting and identifying the multivalent molecules that bind the sequencing polymerases but do not incorporate into the 3′ end of the primer at steps (c) and (d). In some embodiments, the sequence of the nucleic acid template molecule can be determined (or confirmed) by detecting and identifying the nucleotide that incorporates into the 3′ end of the primer at steps (h) and (i).

In some embodiments, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 9-12.

In some embodiments, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. Exemplary multivalent molecules are shown in FIGS. 9-12.

In FIG. 9, the left (Class I) is schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

### Sequencing-by-Binding

Embodiments of the present disclosure provide methods for sequencing any of the immobilized template molecules described herein, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

### Methods for Sequencing Using Phosphate-Chain Labeled Nucleotides

Embodiments of the present disclosure provide methods for sequencing using immobilized sequencing polymerases which bind non-immobilized template molecules, wherein the sequencing reactions are conducted with phosphate-chain labeled nucleotides. In some embodiments, the sequencing methods comprise step (a): providing a support having a plurality of sequencing polymerases immobilized thereon. In some embodiments, the sequencing polymerase comprises a processive DNA polymerase. In some embodiments, the sequencing polymerase comprises a wild type or mutant DNA polymerase, including for example a Phi29 DNA polymerase. In some embodiments, the support comprise a plurality of separate compartments and a sequencing polymerase is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

In some embodiments, the sequencing method further comprises step (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises step (c): contacting the plurality of polymerase/template/primer complexes with a plurality of phosphate chain labeled nucleotides each comprising an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups can be referred to as alpha, beta, and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes are contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiment, the polymerase-catalyzed nucleotide incorporation reaction cleaves between the alpha and beta phosphate groups thereby releasing a multi-phosphate chain linked to a fluorophore.

In some embodiments, the sequencing method further comprises step (d): detecting the fluorescent signal emitted by the phosphate chain labeled nucleotide that is bound by the sequencing polymerase, and incorporated into the terminal end of the sequencing primer. In some embodiments, step (d) further comprises identifying the phosphate chain labeled nucleotide that is bound by the sequencing polymerase, and incorporated into the terminal end of the sequencing primer.

In some embodiments, the sequencing method further comprises step (d): repeating steps (c)-(d) at least once. In some embodiments, sequencing methods that employ phosphate chain labeled nucleotides can be conducted according to the methods described in U.S. Pat. Nos. 7,170,050; 7,302,146; and/or 7,405,281.

### Sequencing Polymerases

Embodiments of the present disclosure provide methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a template molecule. In some embodiments, the sequencing polymerase(s) is/are capable of binding a complementary nucleotide unit of a multivalent molecule opposite a nucleotide in a template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Nucleotides

Embodiments of the present disclosure provide methods for sequencing nucleic acid molecules, where any of the sequencing methods described herein employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine) palladium (0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride. In some embodiments, the chain terminating moiety may be cleavable/removable with nitrous acid. In some embodiments, a chain terminating moiety may be cleavable/removable using a solution comprising nitrite, such as, for example, a combination of nitrite with an acid such as acetic acid, sulfuric acid, or nitric acid. In some further embodiments, said solution may comprise an organic acid.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl) phosphine (TCEP) or bis-sulfo triphenyl phosphine (BS-TPP) or Tri (hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as, for example, formic acid, acetic acid, propionic acid, butyric acid, isobutyric acid, or the like. In some embodiments, the chain terminating moiety comprises a 3′-acetal moiety which can be cleaved with a palladium deblocking reagent (e.g., Pd(0)).

In some embodiments, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety or derivatives thereof.

In some embodiments, in any of the methods for sequencing nucleic acid molecules described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine) palladium (0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl) phosphine (TCEP) or bis-sulfo triphenyl phosphine (BS-TPP) or Tri (hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

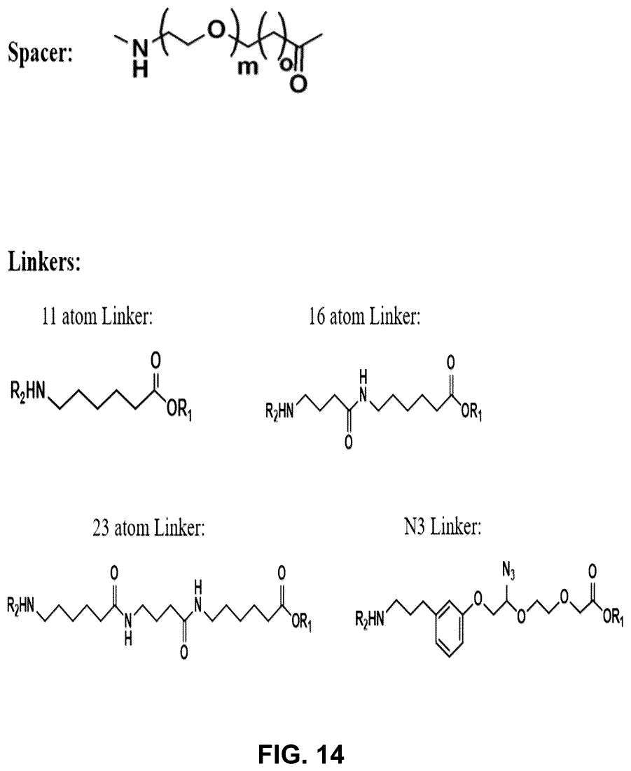

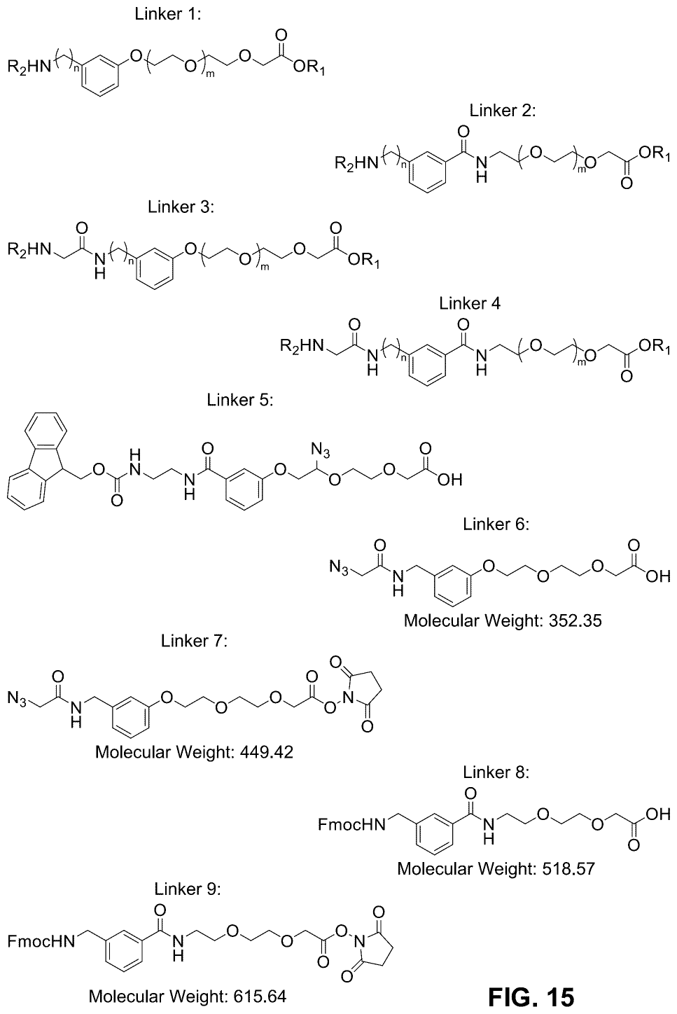

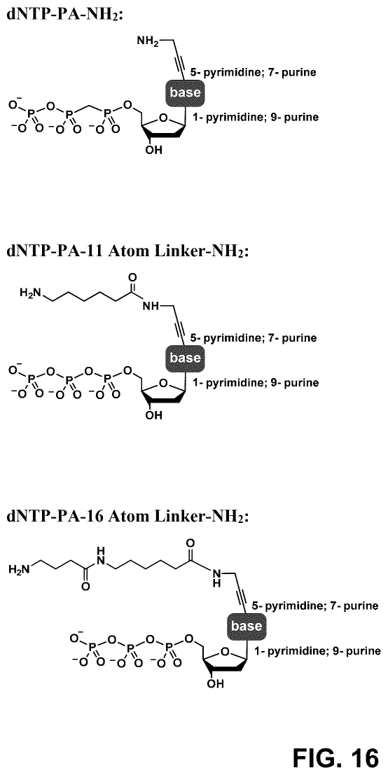

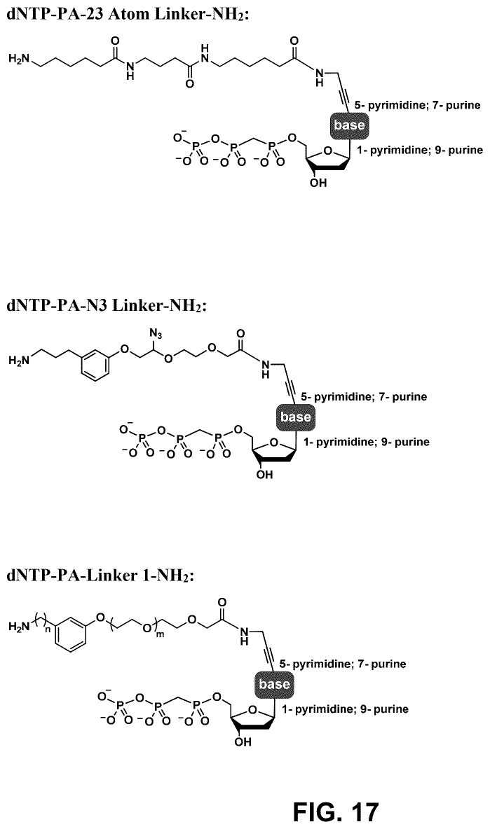

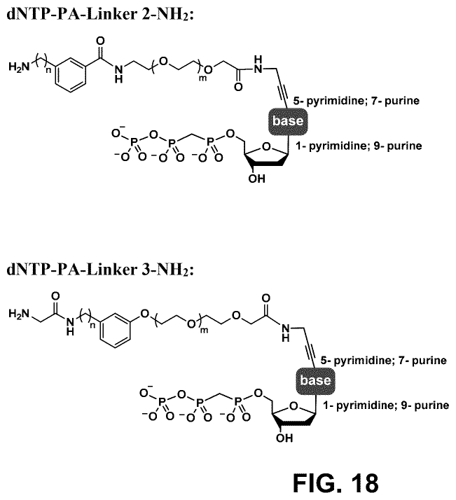

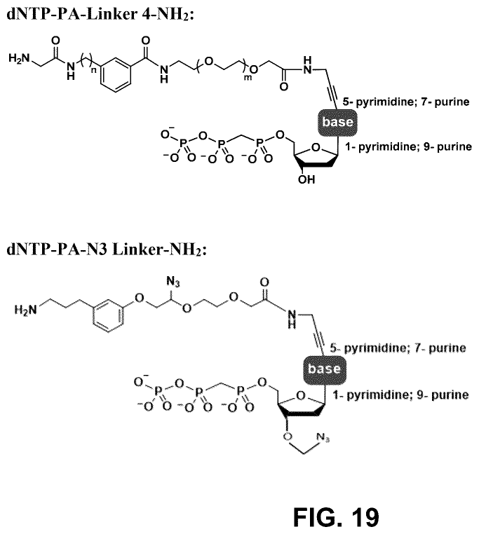

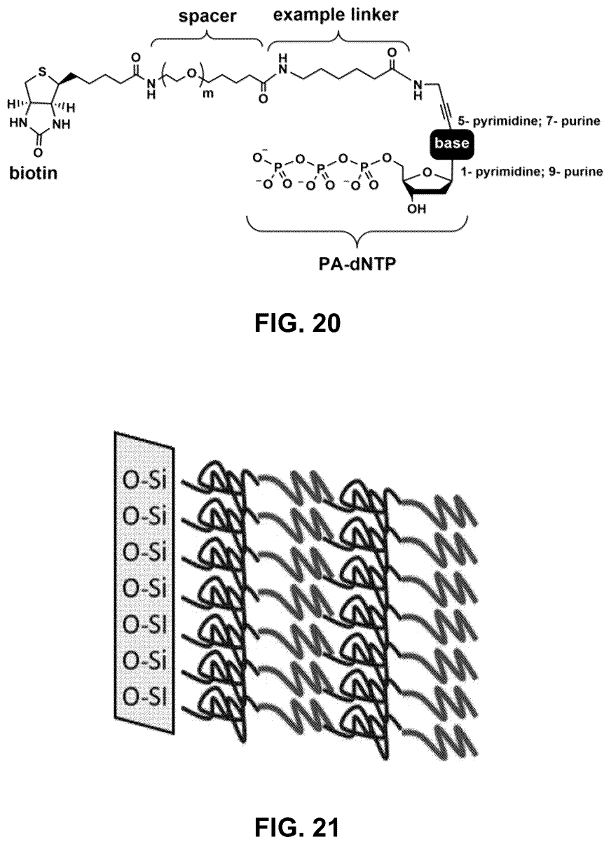

Embodiments of the present disclosure provide methods for sequencing nucleic acid molecules, where any of the sequencing methods described herein employ at least one multivalent molecule. In some embodiments, the multivalent molecule comprises a plurality of nucleotide arms attached to a core and having any configuration including a starburst, helter skelter, or bottle brush configuration (e.g., FIG. 9). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An example nucleotide arm is shown in FIG. 13. Exemplary multivalent molecules are shown in FIGS. 9-12. An example spacer is shown in FIG. 14 (top) and example linkers are shown in FIG. (bottom) and FIG. 15. Exemplary nucleotides attached to a linker are shown in FIGS. 16-19. An example biotinylated nucleotide arm is shown in FIG. 20.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine) palladium (0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl) phosphine (TCEP) or bis-sulfo triphenyl phosphine (BS-TPP) or Tri (hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker, and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the methods for sequencing nucleic acid molecules described herein can include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

A compaction oligonucleotide comprises a single-stranded linear oligonucleotide having a 5′ region that can hybridize to a first portion of a concatemer molecule and the compaction oligonucleotide having a 3′ region that can hybridize to a second portion of the concatemer molecule (e.g., the same concatemer molecule). In some embodiments, hybridization of the compaction oligonucleotides to individual concatemer molecules causes the concatemer molecule to collapse or fold into a DNA nanoball which is more compact in shape and size compared to a non-collapsed DNA molecule. A spot image of a DNA nanoball can be represented as a Gaussian spot and the size can be measured as a full width half maximum (FWHM). A smaller spot size as indicated by a smaller FWHM typically correlates with an improved image of the spot. In some embodiments, the FWHM of a DNA nanoball spot can be about 10 μm or smaller. The DNA nanoball can be a compact nucleic acid structure having a full width half maximum (FWHM) that is smaller compared to a concatemer that is not collapsed/folded into a DNA nanoball.

In some embodiments, compaction oligonucleotides comprise a single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprises a 5′ region and a 3′ region, and optionally an intervening region between 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer molecule. The 3′ region of the compaction oligonucleotides can be wholly complementary or partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule. The 5′ and 3′ regions of the compaction oligonucleotide can hybridize to the concatemer to pull together distal portions of the concatemer causing compaction of the concatemer to form a DNA nanoball.

The 5′ region of the compaction oligonucleotide can have the same sequence as 3′ region. The 5′ region of the compaction oligonucleotide can have a sequence that is different from 3′ region. The 3′ region of the compaction oligonucleotide can have a sequence that is a reverse sequence of 5′ region.

In some embodiments sequence data may be derived through nanopore sequencing, which comprises sequencing of a nucleic acid by translocating said nucleic acid across a membrane, such as through a pore, and wherein sequence reads or base calls are made by measuring one or more signals during the translocation event, such as impedance, current, voltage, or capacitance. In some embodiments, the identity of a nucleotide may be determined by distinctive electrical signatures, such as the timing, duration, extent, or lineshape of a current block, impedance change, voltage change, or capacitance change. Sequencing of nucleic acids by translocation across a membrane and/or through a pore does not foreclose alternative detection methods, such as optical, chemical, biochemical, fluorescent, luminescent, magnetic, electromagnetic, acoustic, or electroacoustic detection.

### Supports and Low Non-Specific Coatings

In some embodiments, the flow cell 112 in FIG. 1 can include a support, e.g., a solid support as disclosed herein. The present disclosure provides pairwise sequencing compositions and methods which employ a support comprising a plurality of oligonucleotide surface primers immobilized thereon. In some embodiments, the support is passivated with a low non-specific binding coating. The surface coatings described herein exhibit very low non-specific binding to reagents typically used for nucleic acid capture, amplification, and sequencing workflows, such as dyes, nucleotides, enzymes, and nucleic acid primers. The surface coatings exhibit low background fluorescence signals or high contrast-to-noise (CNR) ratios compared to conventional surface coatings.

The low non-specific binding coating comprises one layer or multiple layers (FIG. 21). As shown in FIG. 21, in some embodiments, the support comprises a glass substrate and alternating layers of hydrophilic coatings which are covalently or non-covalently adhered to the glass, and which further comprises chemically-reactive functional groups that serve as attachment sites for oligonucleotide primers.

In some embodiments, the plurality of surface primers are immobilized to the low non-specific binding coating. In some embodiments, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl)trimethoxysilane (APTMS), (3-Aminopropyl)triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, surface primers with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a desired surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl) acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 μl deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surface disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some embodiments, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (Signal)-B (interstial) in the FOV) yields a discernable feature that can be classified. In some embodiments, the intrastitial background (B (intrastitial)) can contribute a confounding fluorescence signal that is not specific to the target of interest, but is present in the same ROI thus making it far more difficult to average and subtract.

Nucleic acid amplification on the low-binding coated supports described herein may decrease the B (interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

The headings provided herein are not limitations of the various embodiments of the disclosure, which embodiments can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following embodiments: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever embodiments are described herein with the language “comprising,” otherwise analogous embodiments described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “polony” used herein refers to a nucleic acid library molecule that can be clonally amplified in-solution or on-support to generate an amplicon that can serve as a template molecule for sequencing. In some embodiments, a linear library molecule can be circularized to generate a circularized library molecule, and the circularized library molecule can be clonally amplified in-solution or on-support to generate a concatemer. In some embodiments, the concatemer can serve as a nucleic acid template molecule which can be sequenced. The concatemer is sometimes referred to as a polony. In some embodiments, a polony includes nucleotide strands.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, comprises any enzyme that can catalyze polymerization of nucleotides (including analogs thereof) into a nucleic acid strand. Typically but not necessarily such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One example label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “primer” and related terms used herein refers to an oligonucleotide, either natural or synthetic, that is capable of hybridizing with a DNA and/or RNA polynucleotide template to form a duplex molecule. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-mediated primer extension reaction. Alternatively, 3′ end of the primer can lack a 3′ OH moiety, or can include a terminal 3′ blocking group that inhibits nucleotide polymerization in a polymerase-mediated reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog. The template nucleic acid can be linear, circular, or other forms. The template nucleic acids can include an insert region having an insert sequence which is also known as a sequence of interest. The template nucleic acids can also include at least one adapter sequence. The template nucleic acid can be a concatemer having two or tandem copies of a sequence of interest and at least one adapter sequence. The insert region can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid can be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ⁴²-isopentenyladenine (6iA), N⁶-Δ²-isopentenyl-2-methylthioadenine (2 ms6iA), N⁶-methyladenine, guanine (G), isoguanine, N²-dimethylguanine (dmG), 7-methylguanine (7 mG), 2-thiopyrimidine, 6-thioguanine (6sG), hypoxanthine and 06-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional example bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100:4319-48), acyclic moieties (Martinez, et al., 1999 Nucleic Acids Research 27:1271-1274; Martinez, et al., 1997 Bioorganic & Medicinal Chemistry Letters vol. 7:3013-3016), and other sugar moieties (Joeng, et al., 1993 J. Med. Chem. 36:2627-2638; Kim, et al., 1993 J. Med. Chem. 36:30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6^{th} Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene) penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene) penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene) hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene) hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure can include but are not limited to: nucleotide transient-binding; nucleotide incorporation; deblocking; washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The term “adapter” and related terms refers to oligonucleotides that can be operably linked (appended) to a target polynucleotide, where the adapter confers a function to the co-joined adapter-target molecule. Adapters comprise DNA, RNA, chimeric DNA/RNA, or analogs thereof. Adapters can include at least one ribonucleoside residue. Adapters can be single-stranded, double-stranded, or have single-stranded and/or double-stranded portions. Adapters can be configured to be linear, stem-looped, hairpin, or Y-shaped forms. Adapters can be any length, including 4-100 nucleotides or longer. Adapters can have blunt ends, overhang ends, or a combination of both. Overhang ends include 5′ overhang and 3′ overhang ends. The 5′ end of a single-stranded adapter, or one strand of a double-stranded adapter, can have a 5′ phosphate group or lack a 5′ phosphate group. Adapters can include a 5′ tail that does not hybridize to a target polynucleotide (e.g., tailed adapter), or adapters can be non-tailed. An adapter can include a sequence that is complementary to at least a portion of a primer, such as an amplification primer, a sequencing primer, or a capture primer (e.g., soluble or immobilized capture primers). Adapters can include a random sequence or degenerate sequence. Adapters can include at least one inosine residue. Adapters can include at least one phosphorothioate, phosphorothiolate and/or phosphoramidate linkage. Adapters can include a barcode sequence which can be used to distinguish polynucleotides (e.g., insert sequences) from different sample sources in a multiplex assay. Adapters can include a unique identification sequence (e.g., unique molecular index, UMI; or a unique molecular tag) that can be used to uniquely identify a nucleic acid molecule to which the adapter is appended. In some embodiments, a unique identification sequence can be used to increase error correction and accuracy, reduce the rate of false-positive variant calls and/or increase sensitivity of variant detection. Adapters can include at least one restriction enzyme recognition sequence, including any one or any combination of two or more selected from a group consisting of type I, type II, type III, type IV, type Hs or type IIB.

The term “universal sequence”, “universal adapter sequences” and related terms refers to a sequence in a nucleic acid molecule that is common among two or more polynucleotide molecules. For example, adapters having the same universal sequence can be joined to a plurality of polynucleotides so that the population of co-joined molecules carry the same universal adapter sequence. Examples of universal adapter sequences include an amplification primer sequence, a sequencing primer sequence or a capture primer sequence (e.g., soluble or support-immobilized capture primers).

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support is coated with one or more compounds to produce a passivated layer on the support. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surface disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

Embodiments of the present disclosure provide a plurality (e.g., two or more) of nucleic acid templates immobilized to a support. In some embodiments, the immobilized plurality of nucleic acid templates have the same sequence or have different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support. In some embodiments, the support comprises a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 10²-10¹⁵ sites per mm², or more, to form a nucleic acid template array. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid templates to form a nucleic acid template array. In some embodiments, the nucleic acid templates that are immobilized at a plurality of pre-determined sites by hybridization to immobilized surface capture primers, or the nucleic acid templates are covalently attached to the surface capture primers. In some embodiments, the nucleic acid templates that are immobilized at a plurality of pre-determined sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of pre-determined sites. In some embodiments, individual immobilized nucleic acid template molecules comprise one copy of a target sequence of interest, or comprise concatemers having two or more tandem copies of a target sequence of interest.

In some embodiments, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) are immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates are covalently attached to the surface capture primer. In some embodiments, the nucleic acid templates that are immobilized at a plurality of randomly located sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of randomly located sites. In some embodiments, individual immobilized nucleic acid template molecules comprise one copy of a target sequence of interest, or comprise concatemers having two or more tandem copies of a target sequence of interest.

In some embodiments, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations and/or buffers and the like) onto the support so that the plurality of immobilized nucleic acid template molecules on the support can be reacted with the reagents in a massively parallel manner. In some embodiments, the fluid communication of the plurality of immobilized nucleic acid template molecules can be used to conduct nucleotide binding assays and/or conduct nucleotide polymerization reactions (e.g., primer extension or sequencing) on the plurality of immobilized nucleic acid template molecules, and to conduct detection and imaging for massively parallel sequencing. In some embodiments, the term “immobilized” and related terms refer to nucleic acid molecules or enzymes (e.g., polymerases) that are attached to the support at pre-determined or random locations, where the nucleic acid molecules or enzymes are attached directly to a support through covalent bond or non-covalent interaction, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and it variants refers to a nucleic acid template molecule that has been subjected to one or more amplification reactions either in-solution or on-support. In the case of in-solution amplified template molecules, the resulting amplicons are distributed onto the support. Prior to amplification, the template molecule comprises a sequence of interest and at least one universal adapter sequence. In some embodiments, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

As used herein, the term “sequencing” and its variants comprise obtaining sequence information from a nucleic acid strand, typically by determining the identity of at least some nucleotides (including their nucleobase components) within the nucleic acid template molecule. While in some embodiments, “sequencing” a given region of a nucleic acid molecule includes identifying each and every nucleotide within the region that is sequenced, in some embodiments “sequencing” comprises methods whereby the identity of only some of the nucleotides in the region is determined, while the identity of some nucleotides remains undetermined or incorrectly determined. Any suitable method of sequencing may be used. In an example embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299 (5607): 682-686; Eid, et al., 2009 Science 323 (5910): 133-138; U.S. Pat. Nos. 7,170,050; 7,302,146; and 7,405,281). An example of sequence-by-hybridization includes SOLID sequencing (e.g., from Life Technologies; WO 2006/084132). An example of sequence-by-binding includes Omniome sequencing (e.g., U.S. Pat. No. 10,246,744).

It is to be appreciated that the Detailed Description section, and not any other section, is intended to be used to interpret the claims. Other sections may set forth one or more but not all example embodiments as contemplated by the inventor(s), and thus, are not intended to limit this disclosure or the appended claims in any way.

While this disclosure describes example embodiments for example fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

Additionally, some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

While preferred embodiments of the present invention have been shown and described herein, it will be obvious to those skilled in the art that such embodiments are provided by way of example only. The breadth and scope of this disclosure should not be limited by any of the above-described example embodiments. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A computer-implemented method for adapter trimming in sequencing data analysis, comprising:
acquiring, by a sequencer, flow cell images of one or more sequencing samples;
generating, by a processor, the first sequencing read, the second sequencing read or both based on sequencing analysis of the flow cell images;
selecting, by a processor and based on a plurality of match scores, one or more of:
a first alignment from (a) aligning a tail of a first sequencing read to a head of a second sequencing read at one or more first positions;
a second alignment from (b) aligning the tail of the second sequencing read to the head of the first sequencing read at one or more second positions;
a third alignment from (c) aligning a first adapter to the tail of the first sequencing read at one or more third positions; and
a fourth alignment from (d) aligning a second adapter to the tail of the second sequencing read at one or more fourth positions,
wherein each of the plurality of match scores is based on a first number of matched bases and a second number of total bases;
generating, by the processor, a first consensus position using the first alignment and the third alignment and a second consensus position using the second alignment and the fourth alignment;
determining, by the processor, a trimming position based on the first consensus position, the second consensus position, a first consensus match score, and a second consensus match score; and
trimming, the first sequencing read, the second sequencing read, or both at the trimming position.

2. The computer-implemented method of claim 1, further comprising:
obtaining, by the processor and from the sequencer, the first sequencing read with the first adapter and the second sequencing read with the second adapter,
wherein each of the first sequencing read, the second sequencing read, the head of the first sequencing read, the head of the second sequencing read, the tail of the first sequencing read, the tail of the second sequencing read, the first adapter, and the second adapter comprises a sequence of bases.

3-7. (canceled)

8. The computer-implemented method of claim 1, wherein (a) aligning the tail of the first sequencing read to the head of the second sequencing read at the one or more first positions comprises one of:
aligning (1) a reverse compliment of the head of the second sequencing read to (2) the tail of the first sequencing read at the one or more first positions by shifting the head of the second sequencing read relative to the tail of the first sequencing read by numbers of bases; or
aligning (1) a reverse compliment of the tail of the first sequencing read to (2) the head of the second sequencing read at the one or more first positions by shifting the tail of the first sequencing read relative to the head of the second sequencing read by numbers of bases.

9. (canceled)

10. The computer-implemented method of claim 1, wherein (b) aligning the tail of the second sequencing read to the head of the first sequencing read at the one or more second positions comprises one of:
aligning (1) a reverse compliment of the head of the first sequencing read to the tail of the second sequencing read the one or more second positions by shifting the head of the first sequencing read relative to the tail of the second sequencing read by numbers of bases; or
aligning (1) a reverse compliment of the tail of the second sequencing read to (1) the head of the first sequencing read at the one or more second positions by shifting the tail of the second sequencing read relative to the head of the first sequencing read by numbers of bases.

11-12. (canceled)

13. The computer-implemented method of claim 1, further comprising:
determining, by the processor, the plurality of match scores, wherein each of the plurality of match scores corresponds to an alignment corresponding to one of the one or more first, second, third, and fourth positions.

14. The computer-implemented method of claim 1, wherein the first number of matched bases and the second number of total bases corresponds to one of the one or more first, second, third, and fourth positions.

15. The computer-implemented method of claim 1, wherein generating the first consensus position using the first alignment and the third alignment and the second consensus position using the second alignment and the fourth alignment comprises:
generating a first adapter position based on the first alignment;
generating a second adapter position based on the third alignment;
in response to determining that the first adapter position agrees with the second adapter position, determining the first consensus position as the first adapter position.

16-19. (canceled)

20. The computer-implemented method of claim 1, further comprising:
in response to determining that the first alignment and the third alignment agrees on an adapter position, determining a first consensus match score based on a first sum of matched bases and a second sum of total bases from the first alignment and the third alignment.

21. The computer-implemented method of claim 1, further comprising:
in response to determining that the first alignment and the third alignment disagrees on the first and second adapter positions;
determining a first candidate match score based a first sum of matched bases and a second sum of total bases from a first adapter position obtained from the first alignment and from aligning the first adapter to the tail of the first sequencing read at the first adapter position;
determining a second candidate match score based the first sum of matched bases and the second sum of total bases from a second adapter position obtained from the third alignment and from aligning the tail of the first sequencing read to the head of the second sequencing read at the second adapter position; and
selecting a score from the first and second candidate match scores as the first consensus score.

22. (canceled)

23. The computer-implemented method of claim 1, further comprising:
determining, by the processor, a second consensus match score based on the second alignment and the fourth alignment.

24. The computer-implemented method of claim 1, further comprising:
in response to determining that the second alignment and the fourth alignment agrees on an adapter position, determining a second consensus match score based on a third sum of matched bases and a fourth sum of total bases from the second alignment and the fourth alignment.

25. The computer-implemented method of claim 1 further comprising:
in response to determining that the second alignment and the fourth alignment disagrees on the adapter positions;
determining a third candidate match score based on a third sum of matched bases and a fourth sum of total bases from a third adapter position obtained from the second alignment and from aligning the second adapter to the tail of the second sequencing read at the third adapter position;
determining a fourth candidate match score based the third sum of matched bases and a fourth sum of total bases from a fourth adapter position obtained from the fourth alignment and from aligning the tail of the second sequencing read to the head of the first sequencing read at the fourth adapter position; and
selecting a score from the third and fourth candidate match scores as the second consensus score.

26. (canceled)

27. The computer-implemented method of claim 1, wherein determining the trimming position based on the first consensus position, the second consensus position, the first consensus match score, and the second consensus match score comprises:
selecting a higher score from the first consensus match score and the second consensus match score;
determining that the selected higher score is above a predetermined threshold; and
determining the trimming position based on the selected higher score.

28. The computer-implemented method of claim 1, wherein determining the trimming position based on the first consensus position, the second consensus position, the first consensus match score, and the second consensus match score comprises:
selecting a higher score from the first consensus match score and the second consensus match score;
determining that the selected higher score is below a predetermined threshold; and
determining that no trimming is performed on the first sequencing read or the second sequencing read.

29. The computer-implemented method of claim 1, wherein determining the trimming position based on the first consensus position, the second consensus position, the first consensus match score, and the second consensus match score comprises:
selecting a higher score from the first consensus match score and the second consensus match score;
determining that the selected higher score is above a predetermined threshold; and
determining the trimming position as the first consensus position or the second consensus position, wherein the trimming position corresponds to the selected higher score.

30-68. (canceled)

69. The computer-implemented method of claim 1, further comprising:
converting, by the processor, the trimmed first sequencing read, the trimmed second sequencing read, or both to a predetermined format; and
recording, by the processor, the trimmed first sequencing read, the trimmed second sequencing read, or both in the predetermined format.

70-75. (canceled)

76. A computer-implemented system for adapter trimming in sequencing data analysis, comprising:
one or more hardware processors;
one or more data storage devices storing instructions that, when executed by the one or more hardware processors, cause the one or more hardware processors to perform operations, the operations comprising:
selecting, based on a plurality of match scores, one or more of:
a first alignment from (a) aligning a tail of a first sequencing read to a head of a second sequencing read at one or more first positions;
a second alignment from (b) aligning the tail of the second sequencing read to the head of the first sequencing read at one or more second positions;
a third alignment from (c) aligning a first adapter to the tail of the first sequencing read at one or more third positions; and
a fourth alignment from (d) aligning a second adapter to the tail of the second sequencing read at one or more fourth positions,
wherein each of the plurality of match scores is based on a first number of matched bases and a second number of total bases;
generating a first consensus position using the first alignment and the third alignment and a second consensus position using the second alignment and the fourth alignment; and
determining a trimming position based on the first consensus position, the second consensus position, a first consensus match score, and a second consensus match score.

77-155. (canceled)

156. A computer-implemented method for adapter determination in sequencing data analysis, comprising:
obtaining, by a processor, a plurality of pair-end sequencing reads, each pair-end sequencing read comprising a first and second sequencing read, wherein each of the first and second sequencing read comprise a sequence of nucleotide bases;
determining, by the processor, an adapter position based on:
a first alignment from (a) aligning a tail of a first sequencing read to a head of a second sequencing read at one or more first positions; and
a second alignment from (b) aligning the tail of the second sequencing read to the head of the first sequencing read at one or more second positions;
obtaining, by the processor, a plurality of adapter sequences from the plurality of pair-end sequencing reads based on the determined adapter position;
repetitively performing one or more operations until a stopping criterion is met, comprising:
selecting a seed adapter sequence from the plurality of adapter sequences;
determining one or more adapter sequences among the plurality of adapter sequences that satisfy a similarity threshold in comparison with the seed adapter sequence; and
determining, for each base position, a base of A, G, C, or T/U based on a corresponding count of different bases at the corresponding base position of the one or more determined adapter sequence and a predetermined threshold, thereby generating an individual candidate adapter; and
removing the seed adapter sequence and the one or more determined adapter sequences from the plurality of adapter sequences.

157-158. (canceled)

159. The computer-implemented method of claim 156, wherein determining, by the processor, an adapter position based on: a first alignment from (a) aligning a tail of a first sequencing read to a head of a second sequencing read at one or more first positions; and a second alignment from (b) aligning the tail of the second sequencing read to the head of the first sequencing read at one or more second positions, comprises:
determining, by the processor, the adapter position based on a plurality of match scores, each match score calculated based on a first number of matched bases and a second number of total bases at the one or more first positions and the one or more second positions.

160. (canceled)

161. The computer-implemented method of claim 156, further comprising:
selecting a second seed adapter sequence from the plurality of adapter sequences;
determining one or more second adapter sequences among the plurality of adapter sequences that satisfy a similarity threshold in comparison with the seed adapter sequence; and
determining, for one or more base positions, a base of A, G, C, or T/U based on a corresponding count of different bases at the corresponding base position of the one or more determined adapter sequence and a predetermined threshold; and
in response to determining that n base positions out of a total number of positions fail to satisfy the predetermined threshold, removing the seed adapter sequence and the one or more determined adapter sequences from the plurality of adapter sequences without generating a candidate adapter.

162. The computer-implemented method of claim 156, further comprising:
selecting a second seed adapter sequence from the plurality of adapter sequences;
in response to determining that no adapter sequences among the plurality of adapter sequences that satisfy a similarity threshold in comparison with the second seed adapter sequence, removing the second seed adapter sequence from the plurality of adapter sequences without generating a candidate adapter.

163-211. (canceled)

212. A computer-implemented system for adapter determination in sequencing data analysis, comprising:
one or more hardware processors;
one or more data storage devices storing instructions that, when executed by the one or more hardware processors, cause the one or more hardware processors to perform operations, the operations comprising:
obtaining, by a processor, a plurality of pair-end sequencing reads, each pair-end sequencing read comprising a first and second sequencing read, wherein each of the first and second sequencing read comprise a sequence of nucleotide bases;
selecting, by the processor and based on a plurality of match scores, one or more of:
a first alignment from (a) aligning a tail of the first sequencing read to a head of the second sequencing read at one or more first positions;
a second alignment from (b) aligning the tail of the second sequencing read to the head of the first sequencing read at one or more second positions, wherein each of the plurality of match scores is based on a first number of matched bases and a second number of total bases;
determining, by the processor, an adapter position based on the selected one or more of the first alignment and the second alignment.

213-217. (canceled)
